(19) United States
(12) Patent Application Publication
(10) Pub. No.: US 2025/0295453 A1
(43) Pub. Date: Sep. 25, 2025

(54) SYSTEM AND METHOD FOR SPINAL ALIGNMENT

(71) Applicant: UNIVERSITY HEALTH NETWORK, Toronto (CA)

(72) Inventors: Angela CHEUNG, Toronto (CA); Stephen LEWIS, Toronto (CA); Fatemeh ALAVI, Toronto (CA); Christopher NIELSEN, Toronto (CA); Raja RAMPERSAUD, Toronto (CA)

(21) Appl. No.: 19/084,342

(22) Filed: Mar. 19, 2025

Related U.S. Application Data
(60) Provisional application No. 63/567,093, filed on Mar. 19, 2024.

(51) Int. Cl. A61B 34/10; G16H 20/40; G16H 30/40; G16H 50/30; G16H 50/50

(52) U.S. Cl. CPC A61B 34/10; G16H 20/40; G16H 30/40; G16H 50/30; G16H 50/50; A61B 2034/104; A61B 2034/105; A61B 2034/108

(57) ABSTRACT

Systems and methods for assessing and/or generating a patient-specific alignment for spinal deformity correction surgery are disclosed. A proposed alignment is received. Upon processing at least one pre-operative image of a patient: a plurality of spinopelvic parameters are obtained and at least one computer model of the patient is constructed. An assessment of the proposed alignment is generated using the at least one model. A signal based on the assessment is outputted. A new alignment may be generated based on the results of the assessment.

### CROSS-REFERENCES TO RELATED APPLICATIONS

This application claims all benefit including priority to U.S. Provisional Patent Application No. 63/567,093 filed on Mar. 19, 2024 entitled “SYSTEM AND METHOD FOR SPINAL ALIGNMENT”, the entire contents of which is hereby incorporated by reference.

### FIELD

This disclosure relates to spinal surgery, and more specifically to computer-implemented tools for assisting spinal surgery.

### BACKGROUND

Aging of the population increases musculoskeletal diseases, which significantly impairs the state of health due to sarcopenia, osteoporosis, and arthritis. In the elderly population, the prevalence of back pain is reported up to 75%. Adult spinal deformity (ASD) as a heterogeneous spectrum of abnormalities of the lumbar spine or the thoracolumbar spine, with the prevalence of up to 68%, is increasing in aged societies. Over recent decades, the number of spinal fusion procedures has increased dramatically. The demand for posterior spinal fusion procedures is expected to increase by more than 80% by 2060. While surgery remains the most effective treatment choice, the difficulties arising from surgical complications pose significant challenges. Thus, there is a need to improve such surgeries.

### SUMMARY

In accordance with an aspect, there is provided a computer-implemented method for generating a patient-specific alignment for spinal deformity correction surgery. The method includes upon processing at least one pre-operative image of a patient: obtaining a plurality of spinopelvic parameters; constructing at least one computer model of the patient; computing metrics of muscle expenditure and/or vertebral loading based on the at least one model; and generating a proposed alignment based on the computed metrics.

In this method, the at least one pre-operative image may include a sagittal view image.

In this method, the proposed alignment may include at least one of perfect alignment, an optimal alignment, and a realistic alignment.

This method may further include simulating a plurality of possible sagittal alignments.

In this method, the plurality of possible alignments may correspond to realignment criteria including at least one of Roussouly, SRS-Schwab, age-adjusted, global alignment and proportion (GAP), segmental alignment and T4-L1 axis.

The method may further include generating a prediction of post-operative mechanical complication.

In this method, the post-operative mechanical complication may include at least one of proximal junctional kyphosis (PJK), proximal junctional failure (PJF), distal junctional kyphosis (DJK), and adjacent segment disease.

In this method, the constructing at least one computer model may include fitting a parametric curve onto a spine curvature profile.

In this method, the metrics of vertebral loading may be computed at UIV and UIV+1 levels.

In this method, the metrics of vertebral loading may include metrics of compression and/or shear loading.

This method may further include generating a risk factor for a particular alignment based on finite element analysis applied to the at least one computer model.

In this method, the at least one image may include an x-ray image.

In this method, the at least one image may include bi-planar x-ray images.

In this method, the at least one image may include a CT image.

In this method, the at least one computer model may include a 3D musculoskeletal model.

In this method, the at least one computer model may include a finite element model.

This method may further include presenting a visualization of the proposed alignment.

This method may further include receiving user input corresponding to a desired alignment or a change to the proposed alignment.

This method may further include generating instructions for bending a fusion rod based on the proposed alignment.

This method may further include generating instructions for determining screw type and placement based on the proposed alignment.

This method may further include computing a pelvic compensation.

This method may further include generating at least a portion of a surgical plan.

In accordance with another aspect, there is provided a computer-implemented system for generating a patient-specific alignment for spinal deformity correction surgery. The system includes at least one processor; memory in communication with the at least one processor; software code stored in the memory. The software code when executed at the at least one processor causes the system to: upon processing at least one pre-operative image of a patient: obtain a plurality of spinopelvic parameters; construct at least one computer model of the patient; compute metrics of muscle expenditure and/or vertebral loading based on the at least one model; and generate a proposed alignment based on the computed metrics.

In accordance with a further aspect, there is provided a computer-implemented method for assessing a patient-specific alignment for spinal deformity correction surgery. The method includes receiving a proposed alignment for a patient; upon processing at least one pre-operative image of the patient: obtaining a plurality of spinopelvic parameters; and constructing at least one computer model of the patient; generating an assessment of the proposed alignment using the at least one model; and outputting a signal based on the assessment.

In this method, the generating the assessment may include computing metrics of muscle expenditure and/or vertebral loading for the proposed alignment based on the at least one model.

In this method, the generating the assessment may include generating a risk factor for a particular alignment.

In this method, the outputting the signal may include presenting at least a portion of the assessment to a user via a user interface, such as displaying it on a screen.

This method may further include generating a modification to the proposed alignment based on the signal.

In a yet further aspect, there is provided a computer-implemented system for assessing a patient-specific alignment for spinal deformity correction surgery. The system includes at least one processor; memory in communication with the at least one processor; and software code stored in the memory. The software code when executed at the at least one processor causes the system to: receive a proposed alignment for a patient; upon processing at least one pre-operative image of the patient: obtain a plurality of spinopelvic parameters; and construct at least one computer model of the patient; generate an assessment of the proposed alignment using the at least one model; and output a signal based on the assessment.

Many further features and combinations thereof concerning embodiments described herein will appear to those skilled in the art following a reading of the instant disclosure.

### BRIEF DESCRIPTION OF THE DRAWINGS

In the figures,

FIG. 1 is a schematic diagram of an alignment processing system, in accordance with an embodiment;

FIG. 2 is a schematic diagram of a design subsystem of the alignment processing system of FIG. 1, in accordance with an embodiment;

FIG. 3 depicts an example musculoskeletal model and example spinopelvic parameters, in accordance with an embodiment;

FIG. 4 is a schematic diagram of an assessment subsystem of the alignment processing system of FIG. 1, in accordance with an embodiment;

FIG. 5 depicts three pre-planning criteria: Roussouly, age-adjusted, and GAP for a typical patient with low PI with the illustrated actual preoperative alignment, in accordance with an embodiment;

FIG. 6 depicts graphs showing compression and shear loading at different levels, in accordance with an embodiment;

FIG. 7 depicts a graph showing a relation between PJK and shear loading, in accordance with an embodiment with all the PJK points located inside the large dotted circle and all the non-PJK points located inside the smaller circle at the bottom left corner;

FIG. 8 depicts operation of the alignment processing system, in accordance with an embodiment;

FIG. 9 is a schematic diagram of a computing device, in accordance with an embodiment;

FIG. 10 depicts the position of 10 landmarks collecting from coronal and sagittal EOS images, in accordance with an embodiment;

FIG. 11 depicts an algorithm to update sacrum position based on changes in LL radiographic parameters, in accordance with an embodiment;

FIG. 12 depicts a schematic of the modification of non-instrumented segment (control points represented by stars) according the changes in UIV angle, in accordance with an embodiment;

FIG. 13 depicts creation of a musculoskeletal model from an EOS image: (a) coronal view, (b) sagittal view of EOS image, (c) a Bezier curve fitted to intervertebral discs' centers, (d) a musculoskeletal model represented without muscles, ribs, upper extremities, and pelvis, in accordance with an embodiment;

FIG. 14 depicts postoperative sagittal EOS images of four patients without PJK, in accordance with an embodiment;

FIG. 15 depicts FE prediction of vertebral fracture. (a) Preoperative, (c) Postoperative, and (b) FE predicted failed elements of UIV+1, in accordance with an embodiment;

FIG. 16 depicts the sagittal alignment of a typical patient (a) pre- and (b) postoperatively (c) FE predicted failed elements of UIV+1 under oriented calculated vertebral loading, in accordance with an embodiment;

FIG. 17 depicts the demonstration of two PJK and non-PJK cases with their demographic data, pre and postoperative shear and compression with their load ratios, in accordance with an embodiment;

FIG. 18 depicts location of C7 as a representative of unfused segment end according to C2-FH axis, in accordance with an embodiment;

FIG. 19 depicts a preoperative musculoskeletal model, as well as original and optimal models based on different alignment criteria for a typical patient. (a) preoperative model, (b) and (c) original and optimal models based on Roussouly and segmental alignment, (d) and € original and optimal models based on age-adjusted alignment, (f) and (g) original and optimal models based on GAP, in accordance with an embodiment;

FIG. 20 depicts two main reasons for PJK development. (a) and (b) pre and postoperative images of proximal junction for patients with anteriorly oriented UIV and UIV+1. (c) and (d) pre and postoperative images of proximal junction for patients with PNR (e) an algorithm of PJK development according to the mentioned reasons, in accordance with an embodiment;

FIG. 21 depicts a schematic and formulation to achieve a zero shear alignment, which shows a relation between UIV+1 location and orientation, in accordance with an embodiment;

FIG. 22 depicts analysis for five selected patients with different PI values and TK Cobb angles, (a) Preoperative and postoperative EOS images, (b) The optimal alignments with the actual postoperative UIV orientations, (c) The optimal sets of UIV location and orientation, (d) UIV orientation-location correlations, in accordance with an embodiment;

FIG. 23 and FIG. 24 each is a flowchart showing example operation of the alignment processing system of FIG. 1, in accordance with an embodiment; and

These drawings depict exemplary embodiments for illustrative purposes, and variations, alternative configurations, alternative components and modifications may be made to these exemplary embodiments.

### DETAILED DESCRIPTION

Disclosed herein are systems and methods for computer-implemented processing of patient-specific spinal alignments to aid spinal surgery. In some embodiments, a patient-specific alignment is generated. Such patient-specific alignment may, for example, be generated with the aim of mitigating the risk of postoperative mechanical complications. In some embodiments, proper sagittal alignment in terms of possibility of mechanical complication incident is assessed.

Multiple factors contribute to adverse surgical outcomes. Surgical and alignment variables also can predispose patients to conclude with unsuccessful outcome. Pre-to-postoperative radiographic parameter changes, along with number and levels of instrumented vertebrae, and patient BMI and muscle strength result in different vertebral loading. Depending on the strength of vertebrae and surrounding muscles, influenced by age, sex, and BMD, excessive vertebral loading can cause compression fracture. Proximal junctional kyphosis (PJK) and adjacent segment disease are the most frequent mechanical complications that usually occur following spinal deformity correction surgery. The multifactorial nature of PJK makes it difficult to find its etiology and predict its development. Despite many successes to mitigate PJK by applying different realignment criteria including Roussouly classification, global alignment, and proportion (GAP) score, Scoliosis Research Society (SRS)-Schwab classification, age-adjusted alignment, and segmental alignment, some drawbacks were also addressed. Considering that PJK may or may not develop in patients with the same global sagittal alignment, the possibility of local driver for PJK development is highlighted. Such that more posterior inclination and location of UIV were addressed as profound risk factors for PJK.

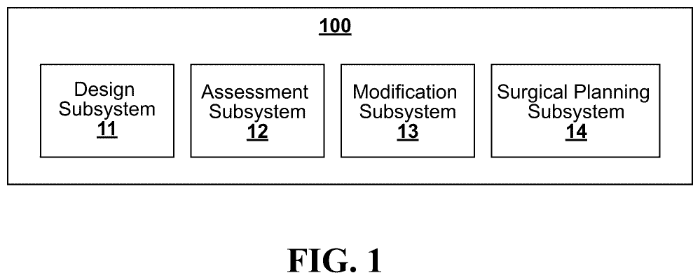

FIG. 1 is a schematic diagram of an alignment processing system 100, in accordance with an embodiment. As depicted, alignment processing system 100 includes a design subsystem 11, an assessment subsystem 12, a modification subsystem 13, and a surgical planning subsystem 14.

In some embodiments, one or more of these subsystems may be omitted, to implement the functionality of a subset of the subsystems. In some embodiments, these subsystems operate in concert as detailed herein.

Design subsystem 11 is configured to generate a patient-specific alignment based on preoperative x-ray images of a patient. In some embodiments, design subsystem 11 generates an alignment that may reduce mechanical loading at upper-instrumented vertebra (UIV) and a level above (UIV+1) in patients undergoing deformity correction surgery. In some embodiments, design subsystem 11 generates an alignment that may reduce muscle expenditure and/or risk of mechanical complications incident.

In some embodiments, design subsystem 11 generates two alignments: a perfect alignment with the minimal muscle expenditure, and an optimal sagittal alignment with minimal vertebral loading (especially shear forces calculated on the superior and interior surface of each vertebra, which mostly are not aligned, such that they provide a shear couple that tends to rotate vertebra over its inferior adjacent one). In some embodiments, the generated alignment may combine a perfect alignment with minimal muscle activation/expenditure in the cone of economy and an optimal alignment with minimal vertebral loading at the proximal junction (UIV and UIV+1 levels). In some embodiments, the use of this combined alignment may reduce the risk of PJK development. A generated alignment may be used by a surgeon in a surgical pre-planning procedure.

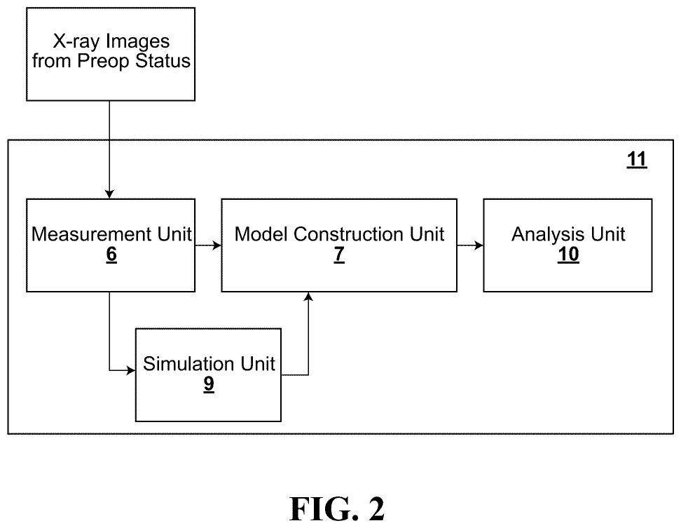

As depicted in FIG. 2, design subsystem 11 includes a measurement unit 6, a model construction unit 7, an alignment simulation unit 9, and an analysis unit 10.

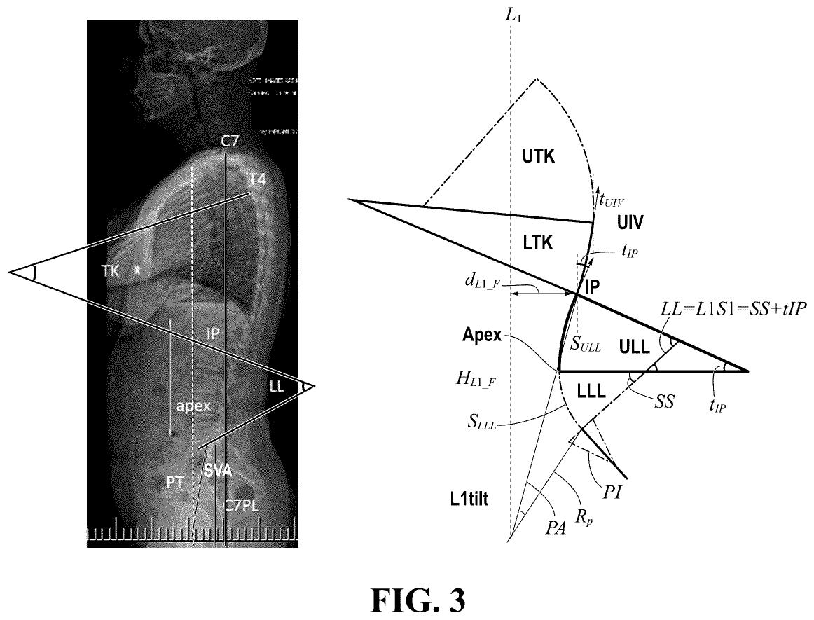

Measurement unit 6 retrieves x-ray images, from which measurements of spinopelvic parameters are obtained. Measurement unit 6 is configured to obtain spinopelvic parameters related to global parameters, regional parameters, and/or segmental parameters. Global parameters may include, for example, pelvic incident (PI), pelvic tilt (PT), sagittal vertical axis (SVA), T1 pelvic angle (TPA), global tilt (GT), mismatch of PI and lumbar lordosis (LL) (PI-LL), sacral slop (SS). Regional parameters may include, for example, LL, thoracic kyphosis (TK), L1-S1, L4-S1; the relative position of each vertebra regarding the pelvis, i.e., vertebral pelvic angle (VPA), and segmental parameters may include Cobb angles between every two vertebrae. Spinopelvic parameters may include, for example, one or more of the parameters depicted in FIG. 3.

In some embodiments, the x-ray images may include images obtained from an EOS imaging system (EOS Imaging, Paris, France), which may be referred to herein as EOS images. In some embodiments, the x-ray images may include images obtained from a system that provides time-synchronized images of two or more planes (e.g., coronal and sagittal planes).

In some embodiments, measurement unit 6 collects additional parameters corresponding to demographic data such as, for example, weight, height, age, sex, or the like.

Model construction unit 7 generates one or more models to assist in the analysis of proposed spinal alignments. In the depicted embodiment, model construction unit 7 includes a musculoskeletal (MSK) modeling subunit that generates musculoskeletal (MSK) models based on x-ray images and a finite element (FE) modeling subunit that generates FE models from CT images.

In some embodiments, the MSK modeling subunit generates a substantially full musculoskeletal model including the thoracolumbar spine, muscles, joints, and the like. In some embodiments, the musculoskeletal model is a three-dimensional (3D) musculoskeletal model, such as, for example, depicted in FIG. 3. In some embodiments, the MSK modeling subunit may utilize demographic data (as may be collected by measurement unit 6). For example, a patient-specific thoracolumbar model may be generated using the sex-matched generic model scaled for height and weight.

Alignment modeling may be performed in sagittal and coronal planes to provide a full 3D musculoskeletal model.

In some cases, the MSK modeling subunit calculates a spinal curvature profile from bi-planar x-ray images. The spinal curvature profile may be a 3D spinal curvature profile. In some cases, the MSK modeling subunit receives a spinal curvature profile, as modified by alignment simulation unit 9.

The MSK modeling subunit identifies the location and orientation of intervertebral discs (IVDs) from preoperative x-ray images. Adjustment of the orientation of the vertebral bodies in the musculoskeletal model is performed. This adjustment uses the patient's spinopelvic parameters, as obtained using measurement unit 6.

In some embodiments, the MSK modeling subunit fits a parametric curve (such as, e.g., a Bezier curve) into the spine curvature profile that smoothens vertebral arrangement and filters out image processing errors of detecting vertebral sequences caused manually or automatically. In some embodiments, this curve fitting may improve the arrangement integrity of the model and may reduce erroneous discrepancies in load calculations.

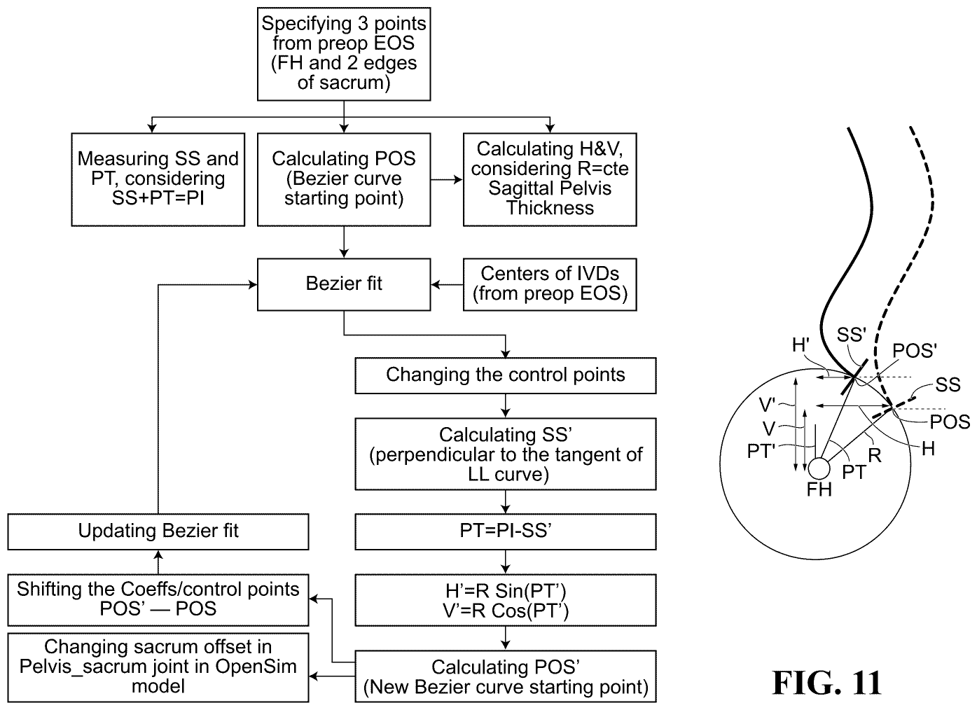

In some embodiments, sacrum position is obtained for one or more calculated alignments. In the depicted embodiment, sacrum position according to sagittal alignment parameter changes is obtained using the algorithm shown in FIG. 11. In some embodiments, upper thoracic compensation (also called upper thoracic reciprocal changes) may be obtained. In some embodiments, other compensations in hip, knee, ankle, or the like may be obtained.

In some embodiments, automated optimization is performed to identify a realistic postoperative sagittal alignment (defined as targeted alignment along with reciprocal changes to satisfy the concept of the cone of economy, which includes changes within the spine, pelvis and lower extremities). In some embodiments, the optimization may include minimizing a cost function directed to minimizing muscle expenditure to maintain the head approximately over the feet.

In some embodiment, optimization parameters may include various compensations including, for example, the amount of pelvic retroversion (PT), knee flexion (KF), and the possible distance of head to the femoral head/feet vertical line (d_head) as a representative of upper thoracic reciprocal changes (UTRCs). The optimization may include generating iteration guesses to alter alignment, and constructing or modifying a corresponding model. Each iteration step may be calculated based on the least square of full body muscle activations. An optimal set of optimization parameters (PT, KF, d_head) will result in the muscle activation least square.

An amount of UTRCs is calculated from the predicted location and orientation of UIV (under the effect of considered PT) and optimized d_head. The OPT procedure may result in a few sets of optimized parameters. Optimized sets may be determined for various realignment schemes including age-adjusted, GAP, segmental and T4-L1 axis, and models with realistic postoperative alignments may be created.

The FE modeling subunit performs image processing analysis to segment the desired bony part of vertebrae from CT images. The FE modeling subunit converts HU units to bone mineral density (BMD) using phantom-less calibration method (based on air, fat, and muscle BMD reference values). The FE modeling subunit creates an FE model and assigns material property to each voxel/element in the model.

Alignment simulation unit 9 simulates proposed alignments, based on different realignment criteria and desired vertebral pelvic angles. Such pre-planning strategies/classifications include for example, Roussouly, SRS-Schwab, age-adjusted, global alignment and proportion (GAP), segmental alignment, and T4-L1 axis, based on which surgeons may perform deformity correction surgery.

Alignment simulation unit 9 may change the parameters of a patient's vertebral curvature profile (e.g., such as a curve as generated by the MSK modeling subunit) based on commonly used realignment schemes. Using optimization procedure, alignment simulation unit 9 matches the vertebral curvature profile to spinopelvic parameters of different commonly used realignment criteria. Targeted parameters in sagittal alignment may include LL, TK, PT, and others.

The positions and orientations (e.g., 6 coordinates) of the vertebrae are determined based on the generated curve. Using this method, the vertebral biomechanical model of spine is generated patient specifically according to various realignment schemes.

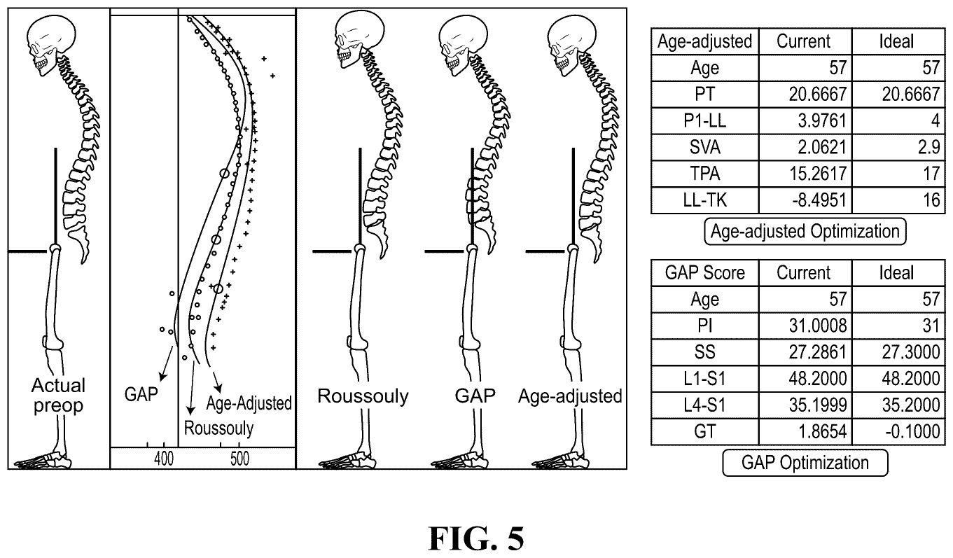

In some embodiments, a fused segment in lumbar and thoracolumbar fusion to the pelvis can also be simulated based on desired regional and segmental parameters like lumber pelvic angle (LPA) and lumbar lordosis (LL). These parameters are used to construct different musculoskeletal models. The calculated alignments based on different sagittal realignment strategies for a typical patient are illustrated in FIG. 5.

Analysis unit 10 is configured to perform MSK and FE analyses. For the MSK analysis, a MSK model based on patient's preoperative status and/or various realignment criteria is analyzed, and vertebral loading at different levels is calculated.

In some embodiments, vertebral loading at different vertebral levels may be calculated at various imaging stages, i.e., preoperative, immediate postoperative, follow-ups. A patient's fixed spinopelvic parameters like pelvic incident (PI) and pelvic radius (PR) using preoperative EOS images is measured. The desired fusion level(s) may be specified by a user.

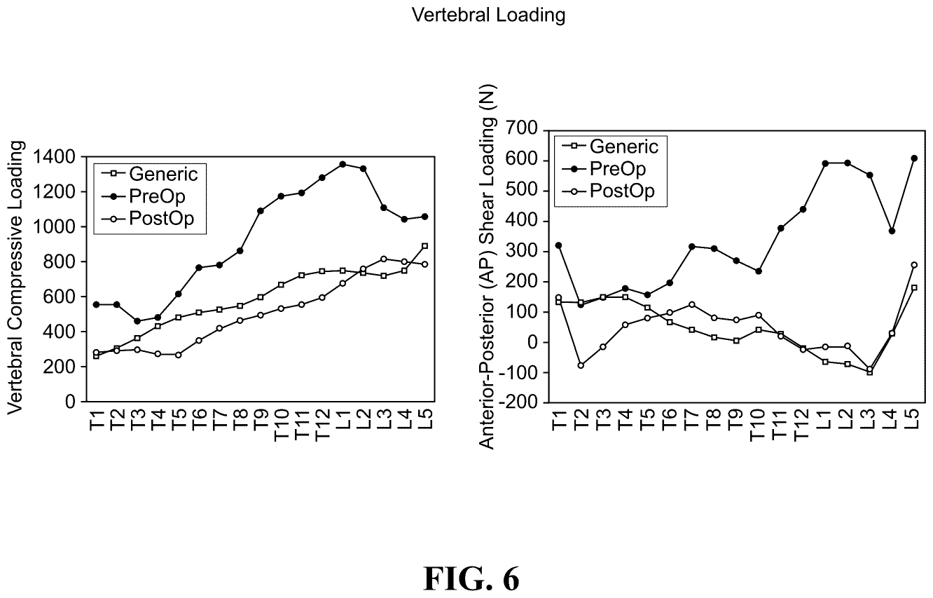

Musculoskeletal analysis includes inverse-dynamic analysis and static optimization procedure to minimize the muscle expenditure which results muscle forces. Then, calculating intervertebral joint reaction forces delivers vertebral loading includes compressive force, shear forces, flexion-extension and mediolateral bending moments, as well as resulted shear couple at each vertebral level. Furthermore, for each alignment muscle expenditure, defined as the square sum of muscle activation, is calculated. A graph consisting compression and shear loading at different levels is shown in FIG. 6.

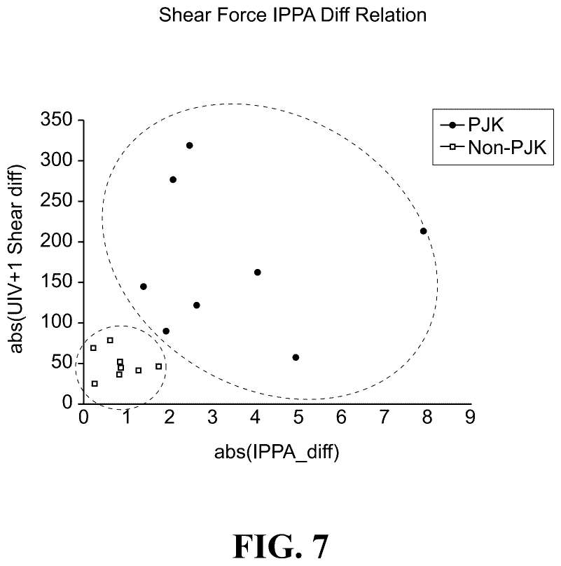

An association between vertebral loading at proximal junction (UIV and/or UIV+1) and the incidence of postoperative mechanical complications like proximal junctional kyphosis (PJK), proximal junctional failure (PJF), distal junctional kyphosis (DJK), and adjacent segment disease has been observed. The plot shown in FIG. 7 illustrates the relation between PJK and shear loading. Although the MSK analysis can be performed independently, the FE analysis can be accomplished following MSK analysis. In the FE analysis section, boundary condition and vertebral loading (shear, compression and bending moment) calculated from MSK analysis is applied to the FE model. The FE analysis is performed to obtain stress and strain fields. Utilizing a proper failure mechanism such as strain energy density and/or maximum strain, the failed elements are identified. A FE risk factor parameter (0<a value<1) is defined for every elements. A risk factor >1 shows the failure of element under the applied force. The average of failed elements' risk factors is used to specify vertebral fracture status. The calculated parameter is a representative of vertebral tolerance according to its BMD and loading situation.

It is believed that UIV+1 bends forward as a result of excessive shear couple on UIV+1 (a couple resulting from shear forces on the superior and inferior surfaces of a vertebra) and the upper thoracic bending moment on UIV. For the sake of instability, an opposite couple has to be created against the shear couple at UIV+1 level. The couple may be created by compressive forces in the following scenario; UIV+1 may rotate adjacent to UIV. This rotation brings it to the anterior edge of UIV and shifts compressive force between UIV and UIV+1. The rotation continues until a couple resulted from the shifted compressive force in the bottom surface of UIV+1 and existing one at the top surface my oppose the UIV+1 shear couple. The explained mechanism is thought to be the main cause of PJK development. The concentrated compressive loading at the anterior superior edge of UIV as well as anterior inferior edge of UIV+1 may result in compression fracture at those regions.

Many believe that BMD is an important risk factor for PJK development. However, the BMD of UIV and UIV+1 do not change pre- to postoperatively, and the existing vertebra even with low BMD is intact preoperatively. An important aspect which may differ post-to-preoperatively is the vertebral force including its magnitude, direction, and its applied point. The vertebral force changes are believed to be determinant in the PJK development. Based on the Wolff's law, the bone aligns and distributes its trabeculae based on the loading status, thus the BMD is distributed to withstand preoperative loading conditions. Dramatic post-to-preoperative changes (in magnitude and direction) of vertebral loading which may not consistent with trabecular orientation and distribution changes in UIV and maybe UIV+1 postoperatively can account for an imperative risk factor in mechanical complication development.

Assessment subsystem 12 is configured to identify the safety of a given alignment. In some embodiments, assessment subsystem 12 determines whether proper alignment was planned for the patient and/or the unfused segment can be well adjusted to the fused corrected segment.

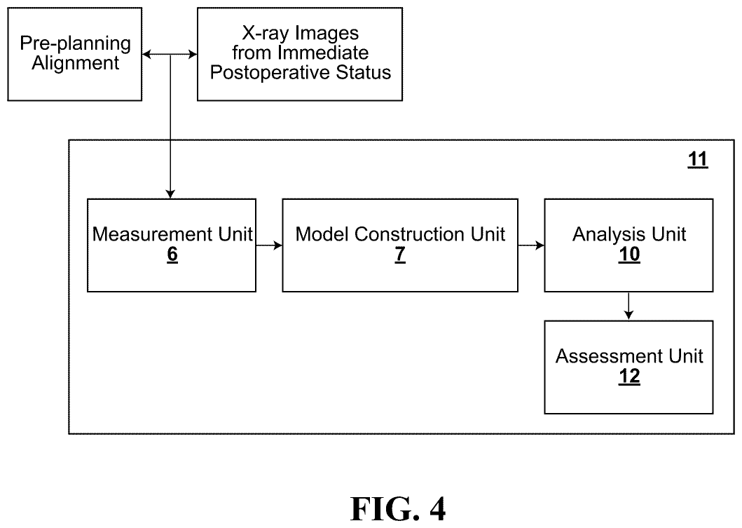

As depicted in FIG. 4, assessment subsystem 12 includes measurement unit 6, model construction unit 7, analysis unit 10, each as described above. In some embodiments, measurement unit 6 receives a preplanned alignment. In some embodiments, measurement unit 6 receives immediate/discharge postoperative x-ray images. Model construction unit 7 determines the applicable parameters and constructs an MSK model. Analysis unit 10 calculates one or more of vertebral loading, muscle expenditure, and an FE risk factor parameter, or other analysis parameters. Assessment unit 12 uses the output of analysis unit 10 to generate a metric predictive of risk of any adjacent segment complication such as, for example, proximal junctional kyphosis (PJK), proximal junctional failure (PJF), distal junctional kyphosis (DJK), or adjacent segment disease.

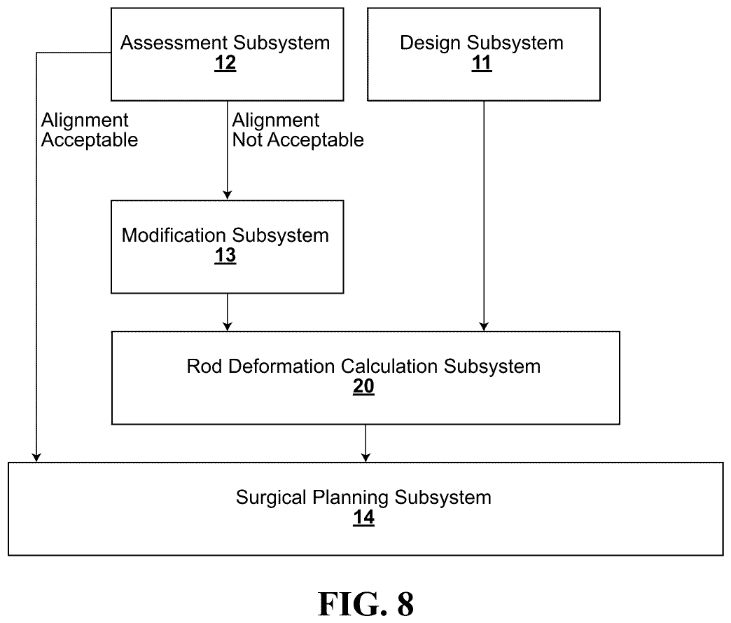

Referring now to FIG. 8, modification subsystem 13 provides a user interface that allows surgeons to input changes/correction in an alignment. In response to such changes/corrections, modification subsystem 13 may provide the corresponding changes in vertebral loading and muscle activation and FE risk factor parameter to surgeons to assist in surgery planning.

Surgical planning subsystem 14 provides a surgeon with a list of alignments consisting of preoperative alignment, simulated alignments according to various realignment schemes, realistic, optimal, and perfect alignments, as well as their demonstration, muscle expenditure, vertebral loading at different vertebral levels, and FE risk factor parameter at UIV and UIV+1 corresponding to that alignment. In some embodiments, realistic alignment includes target spinopelvic parameters according to realignment critera considering reciprical changes (e.g., compensatory mechanism following deformity surgery). In some embodiments, optimal alignment includes sagittal alignment with minimal vertebral forces at proximal junction (adjacent forces). In some embodiments, perfect alignment includes alignment with the minimal muscle expenditure.

Based on the surgeon's selected alignment, surgical planning subsystem 14 is able to compare preoperative and desired alignments and determine a surgical correction procedure to implement the selected alignment. This alignment is imported to a bending apparatus that deforms fusion rod patient-specifically. In some embodiments, surgical planning subsystem 14 may take into account an output of rod deformation calculation subsystem 15, as discussed below.

In some embodiments, surgical planning subsystem 14 includes a reporter that provides visualization of preoperative alignment, different realignment criteria, as well as the realistic, optimal and perfect alignments with their corresponding report of muscle expenditure, adjacent forces and vertebral loading at various levels (e.g., proximal and distal junctions).

In some embodiments, surgical planning subsystem 14 generates a surgical plan for a surgeon-desired alignment and amount of correction needed at proper levels. The surgical plan may be generated based on simulations, such as, for example, by alignment simulation unit 9.

In some embodiments, surgical planning subsystem 14 calculates the amount of vertebral rotation with respect to global axes (X, Y, and Z) as well as Euler angles (local x, y, and z axes) that should performed to reorient each vertebra from preoperative orientation to the desired postoperative one. These calculations help surgeons have an estimation of required rotations for every vertebra, intraoperatively.

In some embodiments, surgical planning subsystem 14 allows surgeon to modify the spinal alignment by controlling either the positions/orientations of one or several vertebrae locally, the global spinopelvic parameters or the global curve parameters. In some embodiments, a graphical user interface is provided to facilitate the described modification procedure.

In some embodiments, surgical planning subsystem 14 allows different simulated models (from design subsystem 11) for a particular patient to be compared not only in terms of sagittal parameters and visual 3D alignment, but also their corresponding vertebral loading at different spinal levels. Such that will help surgeons decide on sagittal alignment based on the minimal vertebral loading at proximal junction.

If the resultant forces are within acceptable range the existing plan will be accepted by assessment subsystem 12, otherwise it will transfer to modification subsystem 13. The revised plan thereafter is sent to surgical planning subsystem 14 to determine the exact surgical procedure to implement the acceptable plan.

A surgical plan based on the difference between preoperative and designed postop alignment is determined. There is a possibility to communicate the predicted surgical correction with users via a display.

In some embodiments, alignment processing system 100 includes a rod deformation calculation subsystem 20. There may be a difference in alignment between an alignment implemented in the operation room and alignment that can be seen in the immediate postoperative x-ray images. This difference may be due to the deformation resulting from the body weight, muscle stiffness, bone fusion, ligament, and joints stiffness. This deformation may be calculated using rod deformation calculation subsystem 20 for particular patients with respect to their demographic data such as trunk's weight and height using a musculoskeletal analysis, which may help achieve an accurate sagittal alignment postoperatively.

Rod deformation calculation subsystem 20 provides the calculated amount of rod deformation to surgical planning subsystem 14. This amount is used to modify a surgical planning alignment to generate an input to a bending apparatus. The bending apparatus is configured to bend the rod based on desired and/or optimal sagittal and coronal alignments. Conveniently, in some embodiments, considering the output of rod deformation calculation subsystem 20 in a preplanned alignment may aid surgeons in achieving a desired alignment.

In some embodiments, surgical planning subsystem 14 proposes the surgical method like posterior column osteotomy (PCO) in which by removing the posterior ligaments (supraspinous, intra-spinous ligaments and ligamentum flavum) and the facet joints, correction is performed through the disc space. In some embodiments, surgical planning subsystem 14 suggests a pedicle subtraction osteotomy (PSO) at specific levels.

In some embodiments, alignment processing system 100 is configured to assess surgical plans. Such surgical plans may be generated at alignment processing system 100 or another system. Such assessment may enable modifications or improvements of surgical plans.

In an embodiment, alignment processing system 100 may assess a surgical plan (and a proposed alignment therein) by generating a prediction of post-operative mechanical complication (e.g., PJK, adjacent segment disease, or the like). The prediction may be generated as a risk metric indicating a likelihood of a complication, such as a risk factor disclosed herein or a risk score. In an embodiment, alignment processing system 100 may assess a surgical plan by computing vertebral loading metrics including, for example, shear forces for the proposed alignment.

In some embodiments, the computed risk metrics and/or loading metrics may be displayed to an operator via a user interface to provide feedback on a surgical plan. In some embodiments, the user interface may be a user interface presented by modification subsystem 13. In such embodiments, the computed risk metrics and/or loading metrics may be updated as the operator makes changes or corrections to a proposed alignment, e.g., using modification subsystem 13. Such updates may be in real-time or quasi-real-time. In such embodiments, the operator may change/correct the proposed alignment until desired metrics are attained, e.g., until the operator confirms that the risk metrics or loading metrics forces are within desired thresholds.

In some embodiments, modification subsystem 13 is configured to automatically suggest modifications to a proposed alignment. Such suggested modifications may be generated, for example, to reduce risk and/or shear forces. Such suggested modifications may be generated, for example, based on simulations of possible alignments (and incremental changes) using simulation unit 9. In some embodiments, modification subsystem 13 is configured to implement the modifications to the proposed alignment automatically. In some embodiments, the modified alignment may be provided to an operator. In some embodiments, the modified alignment may be provided to a bending apparatus to create a patient-specific rod based on the modified alignment.

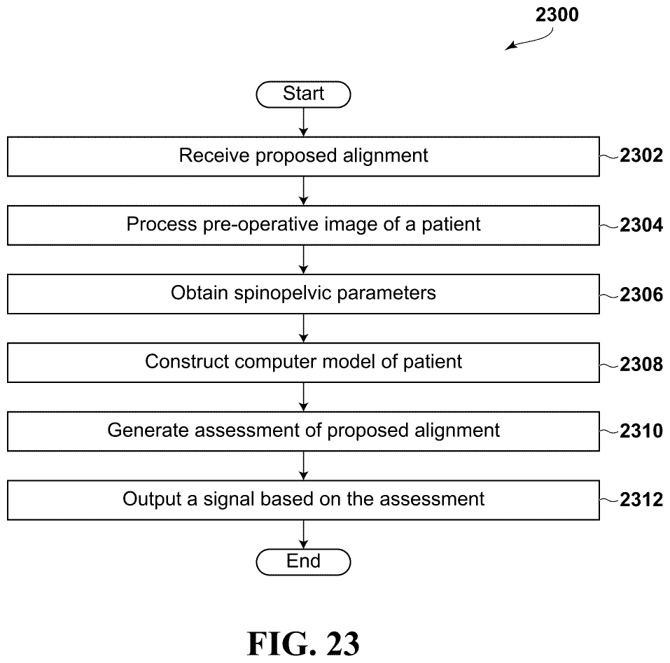

The operation of assessment system 100 is further described with reference to the flowchart depicted in FIG. 23. Assessment system 100 performs the example operations depicted at blocks 2300 and onward, in accordance with an embodiment.

At block 2302, assessment system 100 receives receiving a proposed alignment for a patient, e.g., as part of a surgical plan.

At block 2304, assessment system 100 processes at least one pre-operative image of the patient.

At block 2306, assessment system 100 obtains a plurality of spinopelvic parameters.

At block 2308, assessment system 100 constructs at least one computer model of the patient.

At block 2310, assessment system 100 generates an assessment of the proposed alignment using the at least one model. In some embodiments, generating the assessment may include computing metrics of muscle expenditure and/or vertebral loading for the proposed alignment based on the at least one model. In some embodiments, generating the assessment may include generating a risk factor for a particular alignment.

At block 2312, assessment system 100 outputs a signal based on the assessment. In some embodiments, outputting the signal includes presenting at least a portion of the assessment to a user via a user interface. In some embodiments, a modification to the proposed alignment is generated based on the output signal.

It should be understood that steps of one or more of the blocks depicted in FIG. 23 may be performed in a different sequence or in an interleaved or iterative manner. Further, variations of the steps, omission or substitution of various steps, or additional steps may be considered.

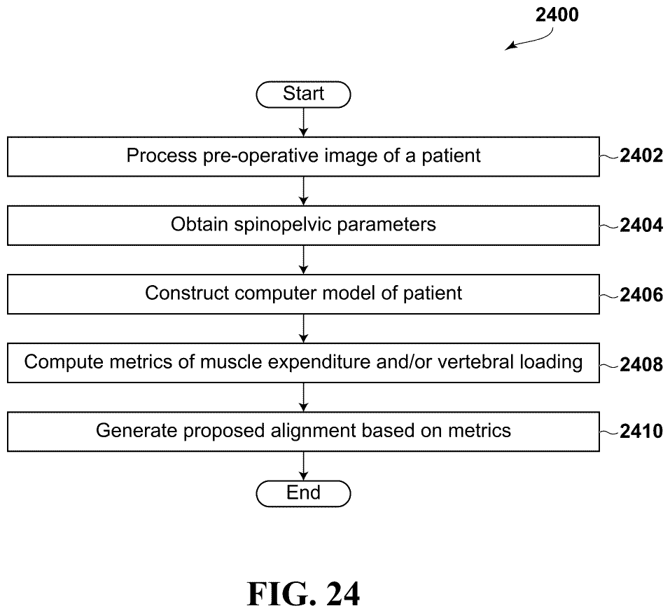

The operation of assessment system 100 is further described with reference to the flowchart depicted in FIG. 24. Assessment system 100 performs the example operations depicted at blocks 2400 and onward, in accordance with an embodiment.

At block 2402, assessment system 100 processes at least one pre-operative image of a patient.

At block 2404, assessment system 100 obtains a plurality of spinopelvic parameters.

At block 2406, assessment system 100 constructs at least one computer model of the patient.

At block 2408, assessment system 100 computes metrics of muscle expenditure and/or vertebral loading based on the at least one model.

At block 2410, assessment system 100 generates a proposed alignment based on the computed metrics.

It should be understood that steps of one or more of the blocks depicted in FIG. 24 may be performed in a different sequence or in an interleaved or iterative manner. Further, variations of the steps, omission or substitution of various steps, or additional steps may be considered.

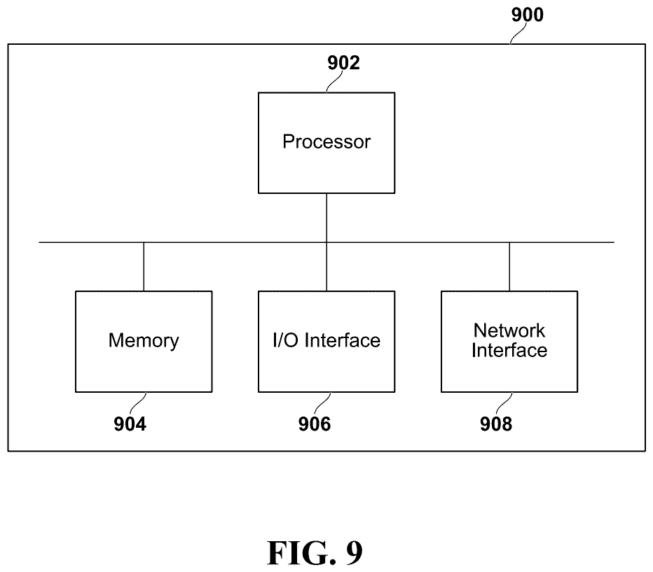

FIG. 9 is a schematic diagram of computing device 900 which may be used to implement alignment processing system 100, in accordance with an embodiment.

As depicted, computing device 900 includes at least one processor 902, memory 904, at least one I/O interface 906, and at least one network interface 908.

Each processor 902 may be, for example, any type of general-purpose microprocessor or microcontroller, a digital signal processing (DSP) processor, an integrated circuit, a field programmable gate array (FPGA), a reconfigurable processor, a programmable read-only memory (PROM), or any combination thereof.

Memory 904 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each I/O interface 906 enables computing device 900 to interconnect with one or more input devices, such as a keyboard, mouse, camera, touch screen and a microphone, or with one or more output devices such as a display screen and a speaker.

Each network interface 908 enables computing device 900 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

For simplicity only, one computing device 900 is shown but assessment system 100 may include multiple computing devices 900. The computing devices 900 may be the same or different types of devices. The computing devices 900 may be connected in various ways including directly coupled, indirectly coupled via a network, and distributed over a wide geographic area and connected via a network (which may be referred to as “cloud computing”).

For example, a computing device 900 may be a server, network appliance, set-top box, embedded device, computer expansion module, personal computer, laptop, personal data assistant, cellular telephone, smartphone device, UMPC tablets, video display terminal, gaming console, or any other computing device capable of being configured to carry out the methods described herein.

### Study 1

### Materials and Methods

### Subjects

Thirty-five ASD patients [average age 65.8 (SD 11.4 yrs.), 24 females (68%)] operated on with a posterior fusion from lower thoracic (T9-T11) to the pelvis and 2-year follow up were included in this retrospective, single center study. Study approval was obtained from the Institutional Review Board (IRB No. 21-5873). Exclusion criteria consisted of patients with spinal deformities derived from ankylosing spondylitis, neuromuscular fracture, infections, or Scheuermann kyphosis. The images of the thoracolumbar spine were acquired in coronal and sagittal planes using EOS imaging system (EOS Imaging, Paris, France) pre- and immediate postoperatively.

Patients were stratified into two groups based on the incidence of PJK at their last follow-up: PJK group and non-PJK group. PJK was defined when the postoperative Cobb angle between lower endplate of the UIV and the upper endplate of the two supra-adjacent vertebrae (UIV+2) is 10 degrees higher than preoperative. Demographic data is shown in Table 1. There was no statistically significant difference in sex, age and bone mass index (BMI) between two groups with the p-values of 0.17, 0.33, and 0.23 respectively.

TABLE 1
Demographic data of patients in PJK and non-PJK groups

| | PJK (n = 19) | non-PJK (n = 16) | p-value |
|---|---|---|---|
| Female sex (%) | 79% | 56% | 0.17 |
| Age at operation | 67.6 ± 8.8 | 63.6 ± 13.8 | 0.33 |
| (year) | | | |
| Bone mass index | 29.5 ± 5.5 | 27.0 ± 6.6 | 0.23 |
| (kg/m²) | | | |

### Musculoskeletal Model

An OpenSim full body musculoskeletal model was used. The model included 111 degrees of freedom and 620 musculotendon actuators including a fully articulated thoracolumbar spine (T1 through L5), with 3 rotational degrees-of-freedom at each intervertebral joint, and ribcage (24 individual ribs and a sternum). Patient-specific models were created by using a full body sex-matched generic model with the scaled height and weight, and the vertebral bodies were adjusted based on the calculated locations and orientations of intervertebral discs (IVDs) from the EOS images through the procedure explained below.

Two musculoskeletal models were created based on the pre- and early/immediate postoperative EOS images, for each patient, and analyzed under upright standing posture. The simulations were carried out in OpenSim software using static optimization by minimizing the overall sum of the squared muscle activation. Then, intervertebral joints loading including compressive and shear forces were computed using joint reaction analysis. Ultimately, resulting vertebral forces from gravity were normalized to the patient's weight.

### 3D Reconstruction of Sagittal Alignment

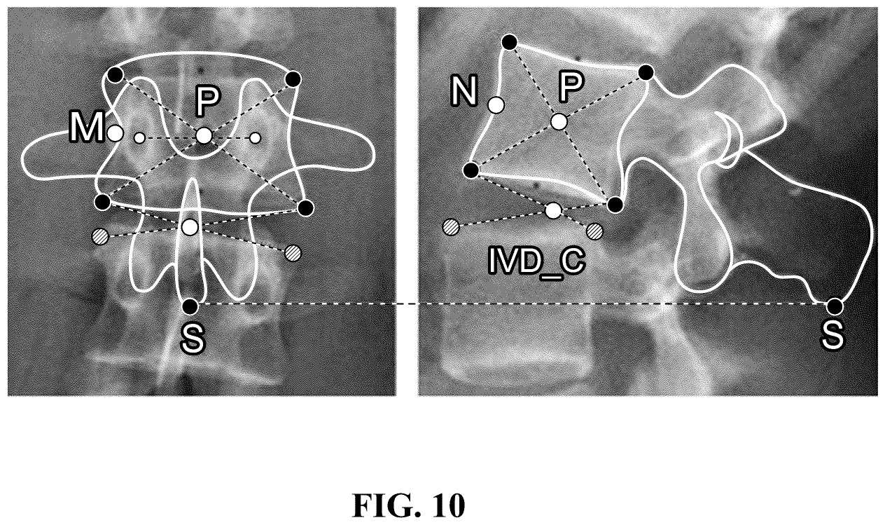

To obtain vertebral and intervertebral location and orientation from bi-planar EOS images, custom MATLAB scripts (MathWorks Inc., Natick, MA, USA) were developed. The one-to-one scale of EOS system allows for acquisition of true to size images; however, converting pixel values to metric dimension was performed using scaling bars at EOS images. The location and orientation of each vertebra was identified using a set of anatomical landmarks including 10 points on the frontal and sagittal planes of bi-planar radiography images as shown in FIG. 10. A Right-hand local coordinate system was defined at each vertebra on its center € as the intersection between the vertebral body diagonals. Using other points including N (the middle point of the anterior edge of a vertebra on sagittal plane), M (the middle point of the left edge of a vertebra on coronal plane), and S (tip of the spinous process), the orientation of coordinate system is obtained such that x-axis is oriented along the projection of PS vector onto the PMN plane; Y-axis is determined by the cross product of PM and PN vectors; finally, the z-axis points toward the cross product of x- and y-axes' unit vectors.

Once the local coordinate systems were determined, the relative rotation of each vertebra with respect to its parent was described by Euler angles as the successive xyz rotations of the local coordinate system with respect to its parent's local coordinate system. In addition, the relative translations of intervertebral joints were calculated, and along with orientations were employed in the OpenSim model.

### Construction of Simulated Mathematical Models

To construct alignment using EOS images or based on targeted sagittal radiographic parameters, a mathematical model was proposed. This tool will help simulate pre-planning alignments based on different classifications or desired spinopelvic parameters. In first step, a Bezier curve was fitted to IVD points collected from the preoperative EOS image. The relative distance of vertebral bodies was stored in the simulated alignment. Then, an optimization procedure was employed to modify Bezier control points to achieve targeted radiographic parameters including segmental, regional, or global sagittal alignments. In addition, actual pre- and postoperative alignments were used to obtain smooth models. The parametric Bezier curve formulae are shown in equations (1) and (2).

$$x ( t ) = {( 1 - t )}^{3} . {x}_{0} + {( 1 - t )}^{2} \text{.3} t . {x}_{1} + ( 1 - t ) \text{.3} {t}^{2} . {x}_{2} + {t}^{3} . {x}_{3} \qquad ( 1 )$$

$$y ( t ) = {( 1 - t )}^{3} . {y}_{0} + {( 1 - t )}^{2} \text{.3} t . {y}_{1} + ( 1 - t ) \text{.3} {t}^{2} . {y}_{2} + {t}^{3} . {y}_{3} \qquad ( 2 )$$

where t is Bezier parameter representative of the points' positions in the curve. xᵢ and yᵢ, i from zero to three, are the horizontal and vertical positions of four Bezier control points, respectively. To determine the control points of Bezier that best fits inside the vertebral points, an optimization algorithm was exploited. The optimization parameters were Bezier control points, and the cost function was the sum of squares of the minimum distances between each IVD point to the Bezier curve. To determine the minimum distance between each IVD point and the Bezier curve, another optimization was used. In the latter optimization algorithm, the optimization parameter was the curve parameter t, and the cost function was the distance between the point and the curve. This optimization was run for every IVD point and the obtained distances were used to construct the former optimization cost function.

Since patients with the fusion from T10 to the pelvis were studied in this study, two cubic Bezier curves fitted into intervertebral joints, one fitted into the curve from sacrum to UIV (T10), the other from UIV to T1. Changing control points of cubic Bezier curves, however, enables us to change sagittal alignment based on desired spinopelvic parameters. Bezier curve fitting and model generation were automated using a custom MATLAB script. As Bezier curve fit was applied for the sagittal plane, it implied negligible lateral shear forces; therefore, anterior-posterior shear and axial compressive forces were included in the simulation.

To implement the pelvic compensation and simulate sacrum adjustment to changes in lower lumbar lordosis (LLL) segment during surgery, an algorithm was developed to modify the position of sacrum (FIG. 11). In this figure, P0S is the starting point of spinal alignment (middle of sacrum). H and V are its horizontal and vertical distances to femoral head (FH), respectively. The parameters related to a modified alignment are distinguished by prime.

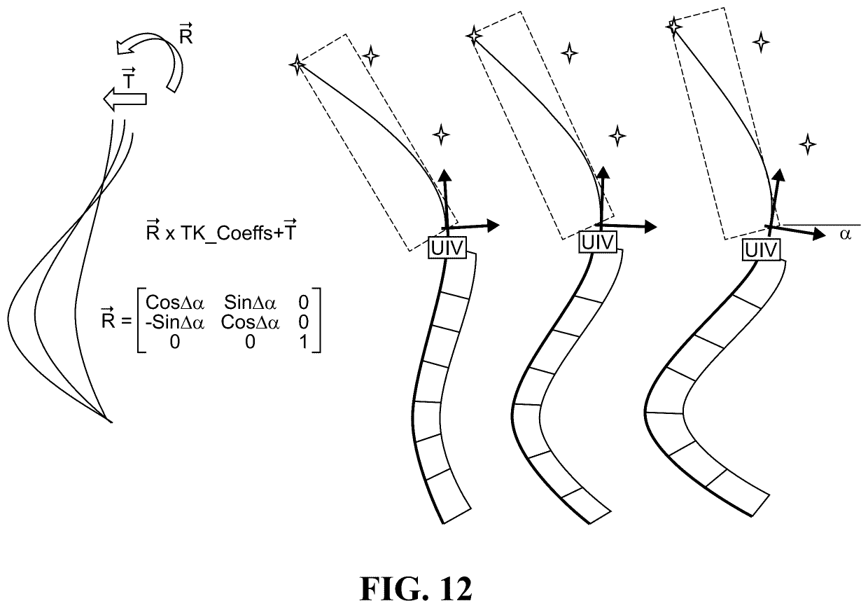

Moreover, the orientation of non-instrumented segment (as an integrated part) must be updated based on the changes in UIV angle, such that the orientation of UIV as the last point of fused Bezier curve and the first point of unfused part should be the same. This procedure was implemented by updating the control points of non-instrumented segment (TK_Coeff) using a translation and rotation matrices as illustrated in FIG. 12, in which Δα is the amount of change in UIV angle of a simulated model compared to actual preoperative one.

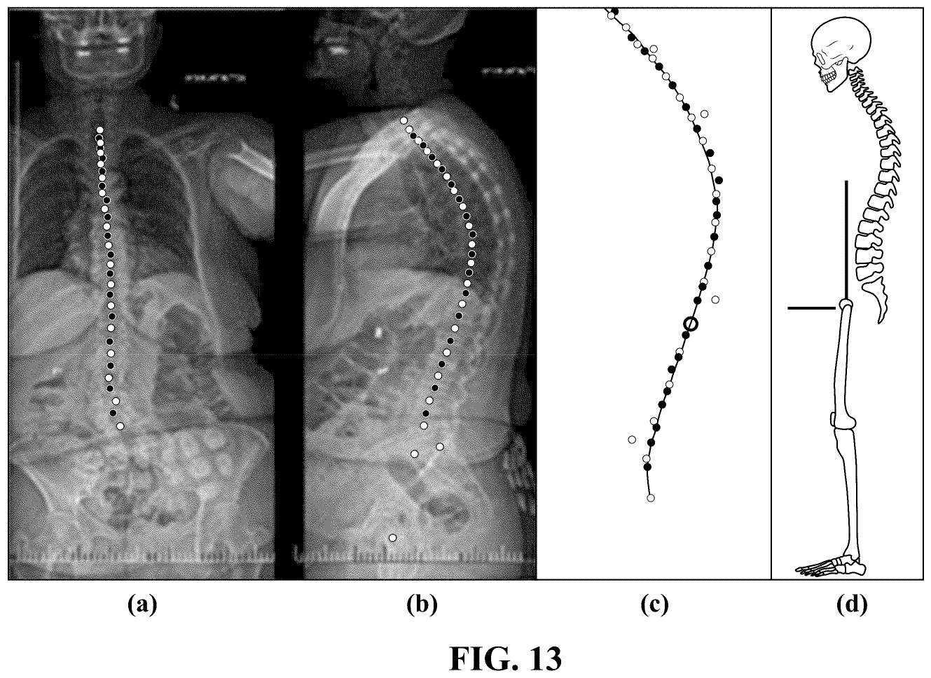

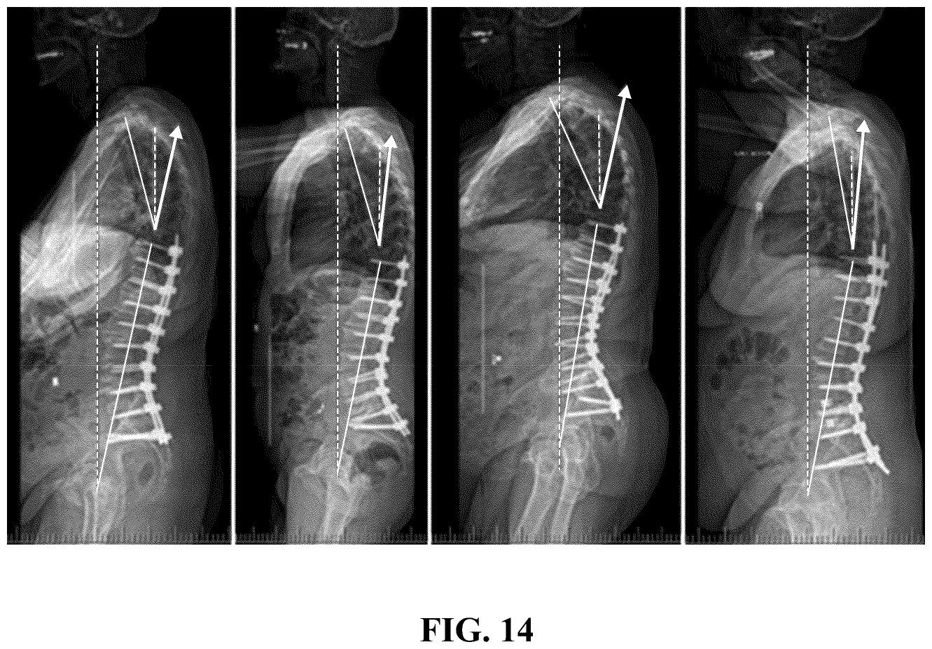

FIG. 13(a) and FIG. 13(b) illustrate coronal and sagittal view of EOS images, respectively. A Bezier curve fitted to intervertebral discs' centers and its corresponding musculoskeletal model are shown in FIG. 13(c) and FIG. 13(d). For the sake of simplicity to illustrate sagittal alignment, the musculoskeletal model was represented without muscles, ribs, upper extremities, and the pelvis. Two types of musculoskeletal models were constructed in this research; models constructed from actual position and orientation of vertebrae in the EOS images (called actual alignment), models built based on a Bezier curve either fitted to the vertebrae within the EOS images or simulate a planned alignment (called simulated alignment). A model based on simulated alignment (so called simulated model) was constructed by locating the control points of Bezier curve such that the sagittal radiographic parameters of actual or planned alignments matched to the simulated alignment. Although the actual and simulated alignments can be compared visually or by comparing Cobb angles between different vertebral levels, a quantitative comparison in terms of vertebral loading was also provided.

### Finite Element (FE) Analysis

Routine CT scans were performed for patients suspected of osteoporosis using a Toshiba Aquilion CT scanner (Toshiba Medical Systems, Tokyo, Japan) with the scanning parameters of 120 kVp tube voltage, 200 mAs adapted tube load. Spine reformations were reconstructed with a slice thickness of 3 mm with a standard bone kernel.

The anonymized clinical pre-operative CT scans of the thoracolumbar spine of patients were analyzed. Depending on the fractured vertebra, UIV or UIV+1, from preoperative CT images was segmented using 3D Slicer software. The segmented vertebra was saved as surface meshes (STL), then the 3D model was meshed using a SegmentMesher extension in 3D Slicer 4.13. After creating tetrahedral (volumetric) meshes, the HU values of nodes were extracted using an extension called “Probe Volume with Model” and python scripts. A 3D FE model was constructed based on the meshed volume. Then, the material property of each element was assigned based on the average value of its four nodes using python scripts. After assigning material property, the boundary condition was applied to the surface at the bottom of the vertebra, representative of the inferior endplate.

The model was loaded with a quasi-static uniformly distributed force calculated from OpenSim musculoskeletal analysis applied to the top surface of a vertebra (representative of superior endplate) perpendicular to the superior endplate toward the caudal direction (compressive force) and tangential direction (anterior-posterior shear force).

Bone was modeled as isotropic and heterogeneous material. The modulus of elasticity for each element was assigned based on the local BMD measured from the CT images. The Hounsfield units were considered equal to BMD equivalent values using a phantomless calibration technique which converts the HU to g/cm³. It was shown that the phantomless calibration based on the combination of air, fat, and muscle provides the highest correlation with respect to the phantom calibration. Thus, multiple slices around UIV with the fat, muscle tissues, and surrounding air were selected as the volume of interest (VOI) using 3D Slicer software. Volumetric measures were extracted opportunistically through the following multi-step procedure. In each scan, the mean HU values of air, fat, and muscle tissues of VOI were obtained using histogram. Then, to obtain the patient-specific air-fat-muscle calibration function, the determined HU values were linearly fitted to the reference BMD values of −840, −80 and 30 corresponding to air, fat, and muscle, respectively. The goodness of fits between BMD and HU were an average R² of 0.99975±0.0005. The slope and intercept were 0.897±0.02 and −1.562±5.88, respectively. The modulus of elasticity for each element was then calculated using experimental equation specific for thoracolumbar vertebrae (Equation (3)):

$$E = 4730 {ρ}_{app}^{1.56} \qquad ( 3 )$$

where ρₐₚₚ was considered equal to BMD. The Poisson's ratio of the vertebra was also considered as 0.3. Moreover, the ash density was obtained through equation (3), and used to calculate strength of each element based on equation (4).

$${ρ}_{ash} = 0.6 \times {ρ}_{app} \qquad ( 4 )$$

$$Strength = \{ \begin{matrix}S = 137 {ρ}_{ash}^{1.88} & {ρ}_{ash} \leq 0.317 trabecular bone \\ S = 114 {ρ}_{ash}^{1.72} & {ρ}_{ash} > 0.317 cortical bone\end{matrix} \} \qquad ( 5 )$$

To predict failure pattern using linear FE analysis, an elemental risk factor (RF) was obtained by computing the ratio of the strain energy density to the yield strain energy density for each element. The failure initiation site was determined based on the locus of the elements with the upmost RF (defined as critical elements) and by increasing the percentage of the screened critical elements, growing failure pattern was simulated. To eliminate the mesh size effect on the results and estimate the optimal mesh size, a mesh convergence study was conducted. The vertebral element size was changed in 3D Slicer and 3D FE model creation and material assignment procedure were repeated.

### Statistical Analysis

Demographic and spinopelvic parameters were described and analyzed in two groups using Excel and Python. Statistical analyses were 2-sided, and the level of significance was set to 0.05. Continuous variables were expressed as mean±standard deviation and their difference between the PJK and non-PJK groups were obtained using an independent t-test. A multivariate logistic regression model was determined using python. For each variable, the odds ratio (OR) with its 95% CI were computed.

### Results and Discussion

### Sagittal Radiographic Parameters

PJK was developed in 19 of 35 (54%) patients until the final follow-up. The pre- and immediate postoperative spinopelvic sagittal parameters for the two groups were measured. The average of sagittal radiographic parameters including pelvic incidence (PI), sacral slope (SS), pelvic tilt (PT), pelvic incidence-lumbar lordosis mismatch (PI-LL), T1-pelvic angle (TPA), global tilt (GT), distal and total lumbar lordosis (i.e. L4-S1 and L1-S1), lumbar pelvic angle (LPA), UIV pelvic angle (UIVPA), non-instrumented segment pelvic angle (C2_UIV+1) measured the angle between two lines connecting C2 to femoral head (FH) and UIV+1 to FH are summarized in Table 2. The preoperative, postoperative, and post-to-preoperative changes radiographic parameters for each group are presented in this Table. PJK patients had significantly higher postoperative TPA and GT, and greater changes between pre and postoperative (p<0.001) in PJK compared to non-PJK patients. There was no statistically significant difference between the PJK and non-PJK groups in the pelvic incidence (PI).

TABLE 2
Comparison of pre- and postoperative sagittal radiographic
parameters between PJK and non-PJK groups

| | | |
|---|---|---|
| PJK Group | non-PJK Group | |
| (n = 19) | (n = 16) | p-values |

Preoperative

| | | | |
|---|---|---|---|
| PI(°) | 55.5 ± 13.9 | 53.8 ± 10 | 0.68 |
| PT(°) | 30.6 ± 11.9 | 26.7 ± 8.2 | 0.26 |
| PI-LL(°) | 35.0 ± 26.0 | 24.9 ± 22.1 | 0.22 |
| TPA(°) | 31.8 ± 12.3 | 25.8 ± 9.3 | 0.11 |
| GT(°) | 40.7 ± 14.9 | 33.1 ± 11.3 | 0.09 |
| L1-S1(°) | 20.4 ± 26.9 | 28.9 ± 22.9 | 0.31 |
| L4-S1(°) | 24.21 ± 10 | 27.8 ± 9.3 | 0.41 |
| LPA (°) | 16.9 ± 8.4 | 15.6 ± 8.8 | 0.67 |
| UIVPA(°) | 19.5 ± 9.6 | 17.7 ± 9.7 | 0.57 |
| C2_UIV + 1 (°) | 11.7 ± 4.6 | 10.5 ± 3.1 | 0.38 |

Postoperative

| | | | |
|---|---|---|---|
| PI(°) | 55.5 ± 13.9 | 53.8 ± 10 | 0.68 |
| PT(°) | 25.0 ± 12.4 | 21.6 ± 6.5 | 0.29 |
| PI-LL(°) | 12.4 ± 1.9 | 14.5 ± 11.0 | 0.95 |
| TPA(°) | 25.5 ± 9.2 | 18.8 ± 6.8 | 0.02* |
| GT(°) | 32.5 ± 11.7 | 24.5 ± 7.9 | 0.02* |
| L1-S1(°) | 43.1 ± 15.8 | 42.9 ± 8.0 | 0.96 |
| L4-S1(°) | 30.3 ± 10 | 29.1 ± 7.8 | 0.69 |
| LPA (°) | 12.9 ± 6.4 | 11.9 ± 5.3 | 0.62 |
| UIVPA(°) | 12.7 ± 7.0 | 11.2 ± 5.8 | 0.51 |
| C2_UIV + 1 (°) | 13.7 ± 4.7 | 10.1 ± 3.1 | 0.01* |

Post-to-preoperative Changes

| | | | |
|---|---|---|---|
| ΔPI(°) | 0.0 | 0.0 | — |
| ΔPT(°) | 7.0 ± 6.4 | 7.9 ± 4.9 | 0.65 |
| Δ(PI-LL)(°) | 23.1 ± 19.3 | 16.8 ± 17.2 | 0.94 |
| ΔTPA(°) | 7.5 ± 5.9 | 8.3 ± 6.6 | 0.7 |
| ΔGT(°) | 9.9 ± 7.0 | 10.4 ± 8.0 | 0.84 |
| ΔL1-S1(°) | 23.5 ± 19.0 | 16.8 ± 17.2 | 0.32 |
| ΔL4-S1(°) | 10.2 ± 10.1 | 11.1 ± 7.4 | 0.78 |
| ΔLPA (°) | −3.9 ± 4.9 | −3.7 ± 6.6 | 0.9 |
| Δ UIVPA(°) | −6.8 ± 7.0 | −6.4 ± 7.2 | 0.87 |
| ΔC2_UIV + 1 (°) | 2.0 ± 2.5 | −0.4 ± 2.1 | <0.001* |

Values: mean ± standard deviation
*significant difference between two groups.
(Δ): changes between postoperative and preoperative parameters
PI: pelvic incidence,
PT: pelvic tilt,
LL: lumbar lordosis,
GT: global tilt
PI-LL: pelvic incidence and lumbar lordosis mismatch
TPA, LPA, UIVPA: T1 pelvic angle, L1 pelvic angle, UIV pelvic angle
C2_UIV + 1: the angle between two lines C2_FH and UIV + 1_FH (FH: femoral head)

### Verification of Sagittal Alignment Simulation

The reliability of mathematical analysis to simulate the sagittal alignment was addressed in this section. To test the accuracy of simulated models, three patients were randomly selected. Two types of musculoskeletal models were constructed from their postoperative EOS images; one model was constructed from actual position and orientation of vertebrae in the EOS images (actual alignment), the other was built based on the Bezier curve fitted to the vertebrae (simulated alignment). The similarity of simulated to actual postoperative alignments not only was verified visually, but also it was studied by comparing the UIV and UIV+1 vertebral loading of the models based on simulated and actual alignments. Compression at UIV (average of the absolute value of compressive loading on the top and bottom surface of UIV) and shear loading at UIV/UIV+1 junction showed no significant difference between models from the simulated and actual alignments (p=0.9) (Table 3).

TABLE 3
Vertebral loading difference between results
from simulated and actual alignments.

| | |
|---|---|
| Compression at UIV | Shear at UIV/UIV + 1 |

| Patients | Actual | Simulated | Actual | Simulated |
|---|---|---|---|---|
| P1 | 127.23 | 133.11 | −28.87 | −28.75 |
| P2 | 229.03 | 241.27 | −52.63 | −53.94 |
| P3 | 279.88 | 286.96 | −196.35 | −206.37 |

### Finite Element Analysis

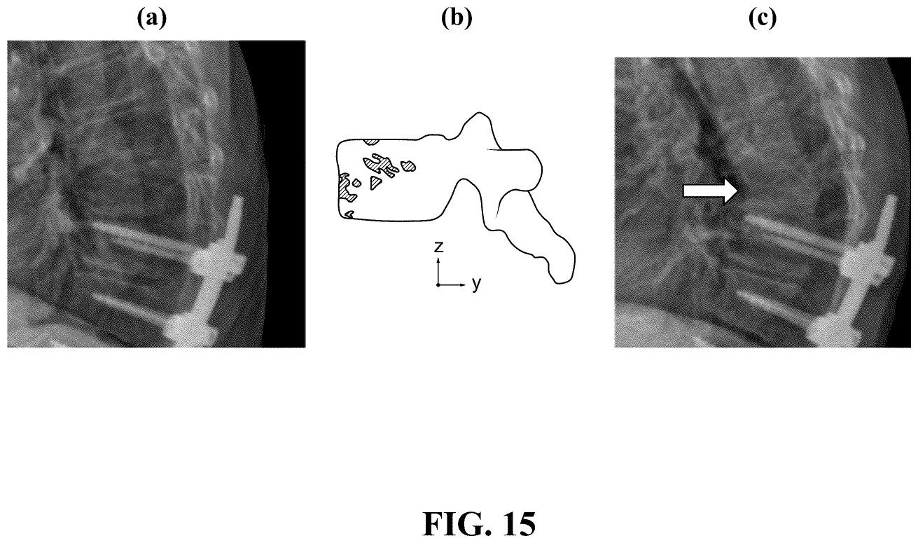

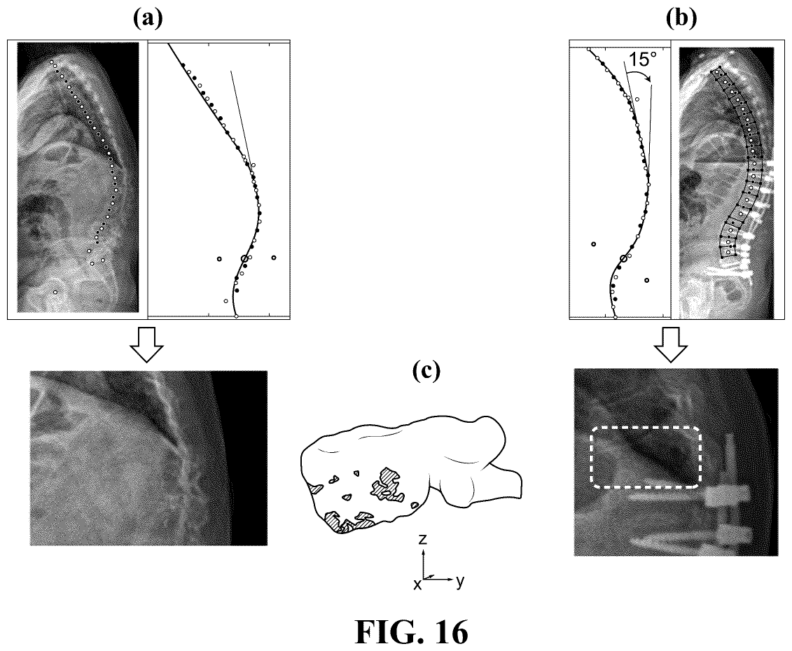

For patients with opportunistic CT scans, FE modeling of UIV or UIV+1 was carried out under their corresponding calculated vertebral loading. The risk factor (RF) was defined by the ratio of the strain energy density to the yield strain energy density for each element. FIG. 15 illustrates intact and failed vertebra at pre- and postoperative EOS images, along with the FE model containing failed elements (with risk factor greater than one), represented in red. The figure shows a perfect agreement between the location of FE-based predicted and actual postoperative failure at UIV+1. For some cases, however, prior to applying calculated postoperative compression and shear forces, the vertebral loading was rotated by the angle of the vertebra's orientation change during the surgery, as preoperative CT scan was used to create a FE model. The procedure for a typical case is shown in FIG. 16. For this patient, the failure occurred at UIV+1. As UIV angle rotated about 15 degrees postoperatively compared to preoperative status, the calculated postoperative shear and compressive forces of UIV+1 were rotated correspondingly, and then applied to the UIV+1 FE model. Again, the predicted failed elements agreed well with the postoperative image.

Association of Vertebral Loading with PJK Development

The vertebral loading including compression and shear forces at UIV and UIV+1 (adjacent forces) was determined using the musculoskeletal analysis for PJK and non-PJK groups. To eliminate the effect of weight in the sagittal alignment-vertebral loading association study, adjacent forces were normalized to the patient's weight. For most alignments, fusion induced shear forces in opposite directions around UIV+1, i.e., anterior shear force at the superior surface and posterior shear force at the inferior surface. These opposing shear forces generate a shear couple (shear forces difference multiply by vertebral width) that contributes to forward bending of UIV+1. Given this observation, we sought to explore the association between this shear couple and PJK development. Additionally, the average of the absolute values of compression on the superior and inferior vertebral surfaces was represented as vertebral compressive force.

The average normalized compression and shear difference at UIV and UIV+1 in PJK and non-PJK groups are summarized in Table 4. The postoperative normalized compression and shear difference, as well as the post-to-pre compression and shear ratio at UIV level were not significantly different between the two groups. However, at UIV+1, the normalized shear difference in PJK group (mean PNSD=−0.25) was 2.5 times compared to non-PJK group (mean PNSD=−0.09) with a p-value of 0.0003. A negative shear difference value demonstrates the counterclockwise direction of the shear couple. Although the UIV+1 post-to-pre shear ratio for PJK group was almost twice compared to non-PJK one, this difference neared statistically significant (p=0.059) in our observation. There was no significant difference between the postoperative normalized compressive force and its post-to-pre ratio between the two groups (p=0.44, 0.51). While PNC values at UIV are significantly different and greater than the ones at UIV+1 in both groups, PNSD values are significantly different and smaller at UIV compared to UIV+1 in both groups.

TABLE 4
Comparison between mean postoperative normalized vertebral
loading and load ratios at UIV and UIV + 1 levels

| | | PJK Group | non-PJK Group | |
|---|---|---|---|---|
| | | (n = 19) | (n = 16) | p-value |
| UIV | PNC | 0.41 ± 0.12 | 0.36 ± 0.02 | 0.13 |
| | PNSD | −0.06 ± 0.11 | −0.025 ± 0.09 | 0.27 |
| | post-to-pre | 0.68 ± 0.25 | 0.69 ± 0.163 | 0.90 |
| | compression | | | |
| | ratio | | | |
| | post-to-pre | 0.97 ± 0.93 | 1.36 ± 1.45 | 0.36 |
| | shear ratio | | | |
| UIV + 1 | PNC | 0.36 ± 0.1 | 0.34 ± 0.02 | 0.44 |
| | PNSD | −0.25 ± 0.15 | −0.09 ± 0.07 | 0.0003** |
| | post-to-pre | 0.73 ± 0.25 | 0.78 ± 0.16 | 0.51 |
| | compression | | | |
| | ratio | | | |
| | post-to-pre | 2.81 ± 2.66 | 1.46 ± 1.26 | 0.059 |
| | shear ratio | | | |

**Statistically significant difference (p < 0.05) between PJK and Non-PJK groups.
Postoperative normalized Compression (PNC) = Compression (N)/weight (N)
Postoperative normalized shear difference (PNSD) = shear difference (N)/weight (N)

Normalized shear and compressive forces were categorized as either high or low, utilizing a threshold of 0.2 and 0.5, respectively. Additionally, the “post-to-pre” load ratio (either shear or compression) is defined as the ratio of the immediate postoperative shear/compression force to the preoperative one, where defined as low (R<1) and high (R>1) ratios. Table 5 outlines four distinct regions based on the postoperative normalized shear and compressive forces and their “post-to-pre” load ratio.

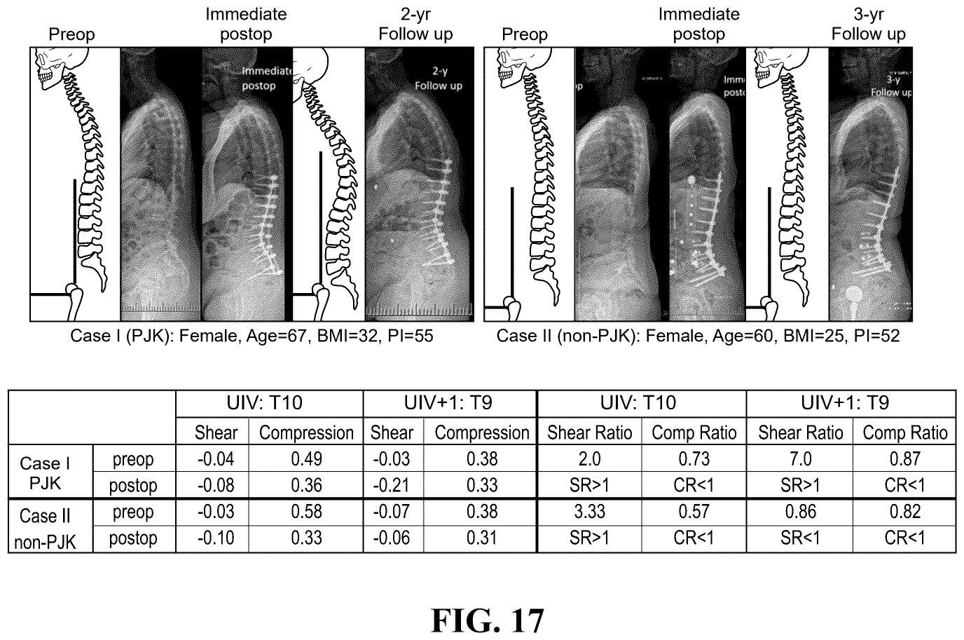

Of 16 non-PJK patients, 15 had postoperative normalized shear difference at UIV+1 (PNSD)<0.2 (Table 4a), and all experienced postoperative normalized compression (PNC)<0.5 (Table 4b). Notably, there were no non-PJK patients found in the regions combining high shear with a high shear load ratio (PNSD>0.2, SR>1), and high compression with high compression ratio (PNC>0.5, CR>1) called high-risk zones. This highlights the importance of lower vertebral loading at the early postoperative stage in preventing the occurrence of PJK. In contrast, these high-risk zones encompassed 14 patients who developed PJK. From other 5 patients in PJK group, 3 experienced higher postoperative shear forces compared to their preoperative ones, despite their low shear values (PNSD<0.2). FIG. 17 demonstrates two PJK and non-PJK cases with their demographic data, pre and postoperative shear and compression with their load ratios.

TABLE 5
Diagram of the number of PJK and non-PK (NPJK) patients based on (a)
normalized shear values and post-to-pre shear ratio, and (b) postoperative
normalized compression values and post-to-pre compression ratio at UIV + 1

| | PNSD > 0.2 | PNSD > 0.2 | | | PNC > 0.5 | PNC > 0.5 | |
|---|---|---|---|---|---|---|---|
| SR < 1 | $\frac{3 PJK}{7 NPJK}$ | $\frac{0 PJK}{1 NPJK}$ | $\frac{3 PJK}{8 NPJK}$ | CR < 1 | $\frac{15 PJK}{14 NPJK}$ | $\frac{1 PJK}{0 NPJK}$ | $\frac{16 PJK}{14 NPJK}$ |
| SR > 1 | $\frac{4 PJK}{8 NPJK}$ | $\frac{12 PJK}{0 NPJK}$ | $\frac{16 PJK}{8 NPJK}$ | CR > 1 | $\frac{1 PJK}{2 NPJK}$ | $\frac{2 PJK}{0 NPJK}$ | $\frac{3 PJK}{2 NPJK}$ |
| | $\frac{7 PJK}{15 NPJK}$ | $\frac{12 PJK}{1 NPJK}$ | $\frac{19 PJK}{16 NPJK}$ | | $\frac{16 PJK}{16 NPJK}$ | $\frac{3 PJK}{0 NPJK}$ | $\frac{19 PJK}{16 NPJK}$ |

| | |
|---|---|
| (a) | (b) |

Postoperative normalized shear difference: PNSD
Post-to-pre Shear ratio: SR
Postoperative normalized compression: PNC
Post-to-pre compression ratio: CR

Thirty of total patients (85%), 16 PJK (84%) and 14 non-PJK (87%), had low post-to-pre compression ratio (Table 4). This indicates that for most patients (85%), compressive loading at the proximal junction decreases postoperatively. However, 68% of patients, 16 PJK (84%) and 8 non-PJK (50%), experienced higher shear force postoperatively. Although fusion decreases compression and increases shear forces at adjacent levels in most cases, the trends cannot be generalized, and it depends on post-to-pre changes in sagittal alignment. Factors such as weight and coronal alignment have an impact on vertebral loading; thereby, to study the vertebral loading changes due to sagittal alignment, we isolated the effect of sagittal alignment by normalizing vertebral loading to patients' weights and disregarding their coronal alignments. This research highlights the postoperative shear value as an imperative risk factor for PJK development and underscores the importance of future work regarding preplanning an alignment with optimized shear values at adjacent levels and mitigate the risk of PJK occurrence, accordingly.

In the analysis of forces at the UIV and UIV+1, it is demonstrated that compressive loading did not change significantly between PJK and non-PJK groups. However, the shear difference at the UIV+1 level was notably higher in the PJK group compared to the non-PJK group. Furthermore, apart from assessing shear status (high/low), there is a rational for post-to-pre load ratio assessment. Osteoporosis, independent of sagittal malalignment, is a well-known risk factor for PJK, however, we believe that studying preoperative loading provides the opportunity to consider BMD and sagittal alignment simultaneously by evaluating the vertebra's load bearing ability based on its preoperative condition. Such that a low post-to-pre ratio indicates safe zone in terms of PJK possibility and specifies the ability to tolerate postoperative loads even in the presence of high shear or compression. As demonstrated in Tables 4a and 4b, 16 out of 19 PJK patients experienced higher postoperative shear than their preoperative one; moreover, 8 and 14 patients out of 16 non-PJK patients were in the low ratio zone (SR<1 and CR<1, respectively). It is worth mentioning that there was no significant difference in sagittal spinopelvic parameters of PJK and non-PJK groups pre and postoperatively, other than TPA, GT, and C2_UIV+1 angle in postoperative status. Therefore, comparing postoperative vertebral forces with preoperative forces provides valuable insights for potentially predicting PJK development by combining the impact of BMD status and adjacent forces resulted from postoperative sagittal alignment.

We demonstrated that postoperative alignment in the high-risk zones characterized by a combination of high shear difference at the UIV+1 level (NSD>0.2) and high shear ratio (SR>1), as well as high compression difference at UIV+1 (NC>0.5) and high compression ratio (CR>1), is strongly associated with the development of PJK. Alignments that do not meet any of high-risk zone criteria were proposed as safe alignments. Incorporating the preoperative loading in the post-to-pre load ratio, makes the high-risk zone criteria patient specific. Using the proposed biomechanically derived criteria, we successfully identified all non-PJK patients (100% specificity) and predicted PJK development in 14 out of 19 patients (73% sensitivity). The moderate outcome for sensitivity suggests that factors other than alignment may have contributed to this adverse event. Furthermore, existing the 3 out of 5 non-predicted PJK patients in the low shear but high ratio zone, may be due to constant load threshold. Further investigation is required to identify the optimal patient-specific threshold which may consider other factors such as muscle weakness.

Anteriorly directed shear force at the superior and posteriorly directed shear force at the inferior surface of UIV+1 cause counterclockwise shear couple (shear difference multiply by vertebral width) at UIV+1 in many cases, which tends to bend the vertebra forward. To maintain equilibrium at this level, an opposing (clockwise) moment in UIV+1 is necessary. This opposing moment is generated by shifted compression under the rotated UIV+1. The rotated UIV+1 on the top of UIV shifts UIV/UIV+1 compressive loading anteriorly, thereby compressive forces above and below of the UIV+1 create a clockwise couple to counteract the shear couple. The forward bending not only increases the Cobb angle between UIV and UIV+1, shifted compressive loading alters the loading scenario around UIV and UIV+1. Depending on the bone mineral density of UIV and UIV+1, it can cause compression fracture at the superior anterior wedge of UIV and inferior anterior wedge of UIV+1; consequently, contribute to the proximal junctional failure (PJF) development. Therefore, shear couple at UIV+1 is one of the crucial contributing factors for PJK development with the mentioned causative mechanism, which can be caused by variety of alignment-related risk factors. These risk factors include LL overcorrection, pelvic non-responsiveness, posteriorly displaced L1 (L1-gravity line distance), and more posterior UIV location from femoral head.

To overcome the UIV+1 shear couple, multiple strategies can be addressed. One of the strategies is ligament reinforcement technology, particularly in cases where damage to the posterior ligament complex results in local instability of the proximal part due to fusion at the UIV level. Strengthening the posterior tension band could prove effective in mitigating the UIV+1 shear couple. Additionally, although yet to be biomechanically defined, the surgical impact (temporary or permanent) on the posterior muscular stabilizers (i.e. dynamic posterior tension band) is also likely at play. Our study presents findings from analyses conducted in the upright standing posture; however, analyses performed in other postures, such as forward bending, revealed increased shear couple at the UIV+1. This increase may be attributed to excessive range of motion at the first non-instrumented vertebra. Additionally, another technique proposed to reduce the risk of PJK occurrence is the utilization of posterior tethering. This technique aims to provide a more gradual transition in range of motion from the fused segment to the non-fused one, potentially reduces shear couple and, consequently, mitigates PJK development.

To overcome the UIV+1 shear couple/sagittal moment, multiple strategies can be addressed. One of the strategies is ligament reinforcement technology, particularly in cases where damage to the posterior ligament complex results in local instability of the proximal part due to fusion at the UIV level. Strengthening the ligaments could prove effective in mitigating the UIV+1 shear couple. Our study presents findings from analyses conducted in the upright standing posture; however, analyses performed in other postures, such as forward bending, revealed increased shear couple at the UIV+1. This increase may be attributed to excessive range of motion at the first non-instrumented vertebra. Additionally, another technique proposed to reduce the risk of PJK occurrence is the utilization of posterior tethering. This technique aims to provide a more gradual transition in range of motion from the fused segment to the non-fused one, potentially resulting in reduced shear couple and, consequently, mitigating PJK development.

However, it is important to highlight that, aside from employing surgical interventions to lower the risk of PJK, focusing on minimizing shear couple through pre-planned sagittal alignment with minimal shear at the UIV+1 level is suggested as a crucial step in PJK prevention. The approach of utilizing Bezier curves to simulate pre-planned alignment and assess shear loading at the proximal junction offers a valuable opportunity to decrease the risk of PJK development. Predicting postoperative alignment has always been essential for identifying postoperative complications. Lafage et al. developed a virtual model, which consists of a combination of postoperative alignment from fused pelvis to UIV-1, preoperative alignment of unfused segment (C2-UIV), and the correction of PT based on the following equation: PT=1.14+0.71×PI−0.52×(maximal lumbar lordosis)−0.19×(maximal thoracic kyphosis). This study, however, tried to predict PJK development and reduce it by describing an ideal spine alignment using a mathematical approach.

Although our study has several unique strengths, it is important to acknowledge several limitations. A primary limitation is the small sample size and the inclusion of patients from two surgeons at a single institution and thus may not be generalizable. To establish the effectiveness of vertebral loading assessment in predicting PJK development, larger cohorts should be investigated. However, it is crucial to note that the primary aim of this study was to demonstrate the feasibility of a musculoskeletal analysis-based approach to determine a causative mechanism for PJK development based on immediate postoperative sagittal alignment. The fact that all analyses were performed under upright standing posture can be addressed as another limitation. A third limitation pertains to the omission of other factors associated with PJK, such as osteoporosis and sarcopenia, in our analysis. These parameters were not considered in this study, which may have implications for the comprehensiveness of our current findings and future work.

### Study 2

### Materials and Methods

### Patients

Twenty patients (average age 65.7 (SD 13.7 yrs); 14 females and 6 males) with upper instrumented vertebra (UIV) of T10 fused to pelvis, with and without PJK were included in this retrospective, single center study. The images of the thoracolumbar spine were simultaneously acquired in coronal and sagittal planes using EOS imaging system (EOS Imaging, Paris, France) pre- and postoperatively. Demographic data is shown in Table 1. There was no difference in age and the body mass index between two groups with p-values of 0.374 and 0.656, respectively.

TABLE 1
Demographic data of patients in PJK and control group

| Variable | PJK | CTRL | p-value |
|---|---|---|---|
| Number of patients | 8 | 12 | |
| Age (year) | 69 | 63.5 | 0.374 |
| Female sex (%) | 100 | 50 | 0.0068 |
| Bone mass index | 30.55 | 29.19 | 0.656 |
| (kg/m²) | | | |

### Pre-Planning Strategies

Planning surgical targets in the sagittal plane are performed using different preoperative planning strategies. Roussouly classification describes four, recently five (+anteverted type 3) types of spinal alignments depending on sacral slope (SS) and the shape of lumbar lordosis (LL), which measured between inflection point and the upper S1 endplate. On the other hand, stratification in an asymptomatic adult population by PI revealed that proximal lordosis increases and apex position moves proximally as PI increases, whereas distal lordosis remained relatively constant, around 35°. The necessity of considering patient-specific lumbar shape rather than simple global lordosis matched to the PI was highlighted. Therefore, an alignment based on the combination of Roussouly and segmental alignments with fixed L4_S1 as 35° was considered, and Roussouly-suggested PI-based lumber apex and inflection point (IP) location, i.e. apex=L4 and IP=L1_L2 for low PI, and apex=L3 and IP=T12_L1 for high PI.

To perform precise preoperative planning, it has been shown to incorporate age into sagittal realignment thresholds and propose some ideal age-specific values for sagittal parameters, such as PT, PI-LL mismatch, SVA, and TPA based on the correlation between preoperative spinopelvic alignment and HRQOL outcomes. The proximal vertebral loading of age-adjusted alignment was studied and compared to other pre-planning alignments. The absence of whole pelvic incidence spectrum and the distribution of lordosis, pelvic anteversion, and negative malalignment as potential causes of mechanical complications in Scoliosis Research Society (SRS)-Schwab classification resulted in a score to better predict these ASD postoperative complications. For each patient, the GAP recommended alignment was created using an optimization procedure.

### Consideration of TK Compensation in Alignment

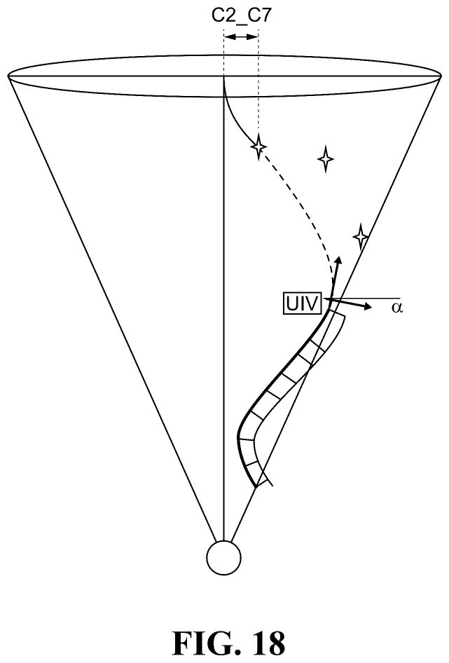

Rigid body rotation and translation can be assumed for unfused segment based on the UIV location and orientation in the fused segment, However, in reality unfused segment is compensated to minimize the location of head above the femoral head. One of the main advantages of the proposed analysis is to consider TK compensation, such that the cranial center of gravity (represented by C2) was assumed to pass through femoral head (creating C2-FH axis) in compensated alignment. Considering constant cervical lordosis pre and postoperatively, the distant between C2 and C7 is measured from preoperative EOS image. Following changes in fused segment based on a pre-planning strategy, automatic changes in Bezier control points of unfused segment are made to achieve the compensated alignment. The Bezier control points are located such that not only unfused part starts from updated UIV location with the corresponding angle, but also C7 locates in the proper distant (C2_C7) from C2-FH axis, as shown in FIG. 18.

### Musculoskeletal Models Constructed Based on Different Alignment Criteria

An OpenSim thoracolumbar full-body musculoskeletal model, consisting of bones, muscles, and joints, was used to calculate vertebral forces at UIV and UIV+1 levels. Sex-matched scaled models were created corresponding to height, and weight. Custom MATLAB scripts were developed to identify location and orientation of intervertebral discs (IVDs) from preoperative EOS images, and used to adjust orientation of vertebral bodies in the model accordingly, as explicitly discussed in our previous publication.

Simulation of alignments based on Roussouly-segmental, age-adjusted, and GAP classifications were completed using a Bezier curve fitted to preoperative EOS image and optimized by changing Bezier control points to match alignment parameters. After constructing patient-specific pre-planned model, musculoskeletal analysis was performed using OpenSim software in the upright standing position to calculate vertebral loading, i.e. shear and compressive loading represented in UIV and UIV+1 local coordinate systems.

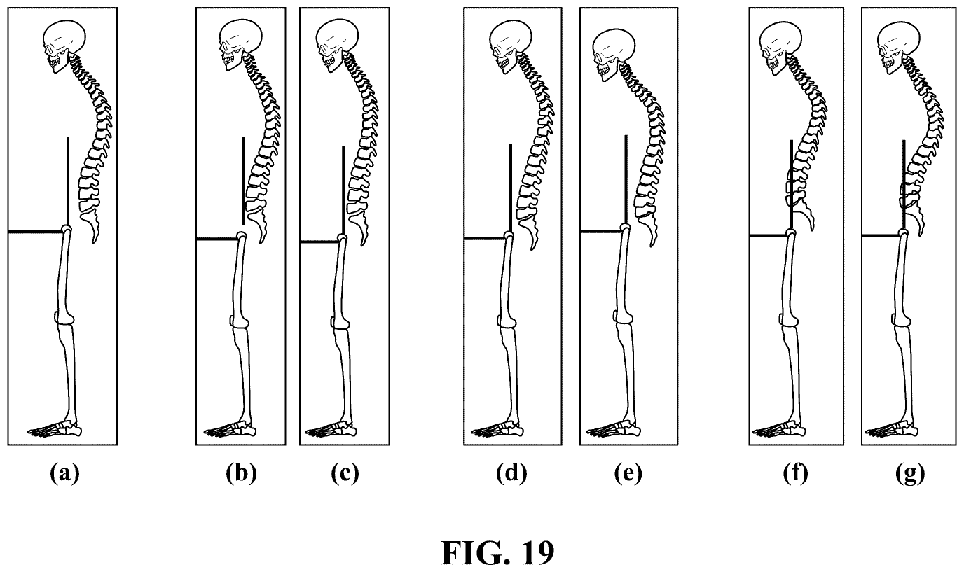

Two series of models according to different alignment criteria were created: original and optimal musculoskeletal models. The original models were constructed based on the target parameters of different alignment criteria, regardless of the optimal UIV location and orientation to reach shear-less condition. While, in optimal models the calculated optimal UIV location and orientation were considered along with the target SS and L4-S1 values based on different strategies. The original and optimal models for one typical patient are illustrated in FIG. 19.

### Statistics

To obtain the differences between UIV/UIV+1 shear loading of alignments based on different classifications, student t-test and ANOVA analysis were performed.

### Results

Analytical explanation

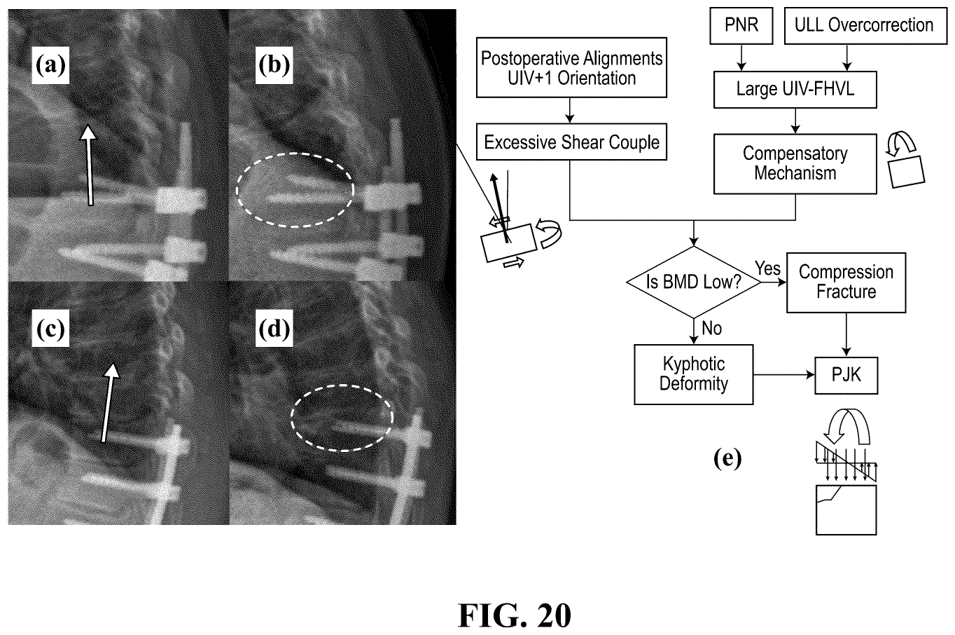

Among the eight patients in PJK group, four had anteriorly orientated UIV+1, which results in excessive shear loading at proximal junction and high shear couple. However, for others long distance of UIV to femoral head (FH) vertical line (UIV-FH) was the main result of increase in the shear force at the proximal junction and PJK development. Large UIV-FH can be caused because of pelvic non-responsiveness (PNR) and/or upper lumbar lordosis (ULL) overcorrection. As UIV was not able to locate in the planned location, the TK compensatory mechanism was triggered to maintain the balanced posture, with the minimal C2 to FH horizontal distance (C2-FH distance) to centered head over the feet. This observation motivated us to develop a numerical solution for considering TK compensation as discussed in section 2.3. Therefore, all analyses in this study were performed by applying TK and pelvic compensation. Two observed reasons (local location and orientation of UIV+1) for PJK development are shown schematically in FIG. 19(e). In this figure, pre and postoperative images of proximal junction for two patients experienced PJK are illustrated. For one patient (FIG. 20(a) and FIG. 20(b)) the main reason of PJK was UIV and UIV+1 orientation, which brought about excessive shear loading, while for the other one (FIG. 20(c) and FIG. 20(d)), large UIV_FH triggered upper thoracic compensatory mechanism, and resulted in PJK accordingly.

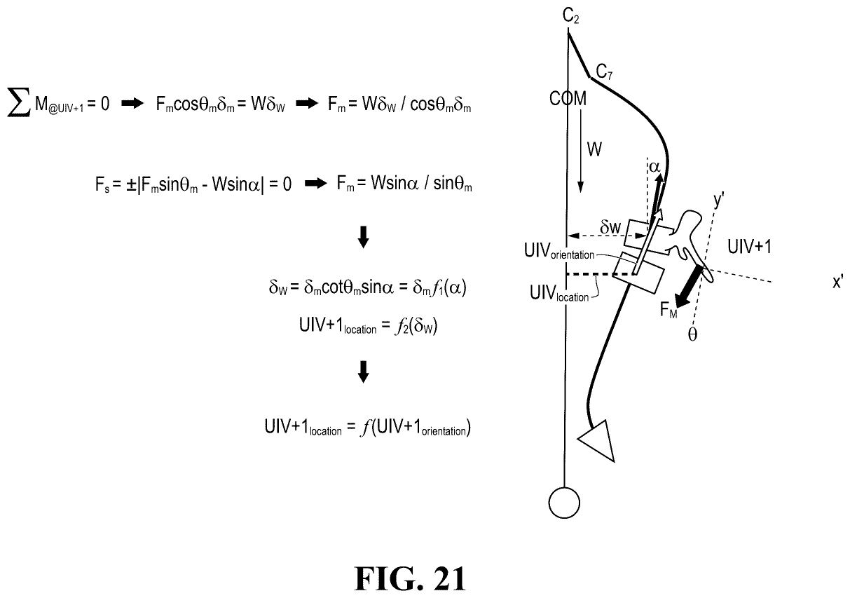

When T1_UIV+1 line and UIV+1 orientation are pointing opposite sides (Type II), there is a chance for minimal shear loading since the weight's component of segment above UIV+1 (Wsinα) can be cancelled out by muscle forces (Fₘsinθₘ) as shown in FIG. 21. This figure shows the schematic and formulation to achieve a zero-shear alignment, which results in a relation between UIV+1 location and orientation. In this figure, W is the weight of head and unfused segment above UIV, δ_{w} is its moment arm to UIV+1, and α is the angle between the line perpendicular to UIV+1 middle surface and the vertical line. Fₘ, δ_{w}, and θ_{w} are the resultant muscle force, its moment arm to the center of UIV+1, and the angle between muscle force direction to UIV+1 direction, respectively. Knowing the fact that UIV+1 orientation is not exactly controllable, we incorporated the effect of UIV orientation on the UIV/UIV+1 shear force, in this study. Seeking an optimal alignment with ideally zero-shear loading analytically illustrates an important result that UIV location and orientation are linearly dependent. This may change our insight to achieve ideal alignment based on modifiable geometrical risk factors.

### Numerical Analysis to Obtain Optimal UIV Location and Orientation

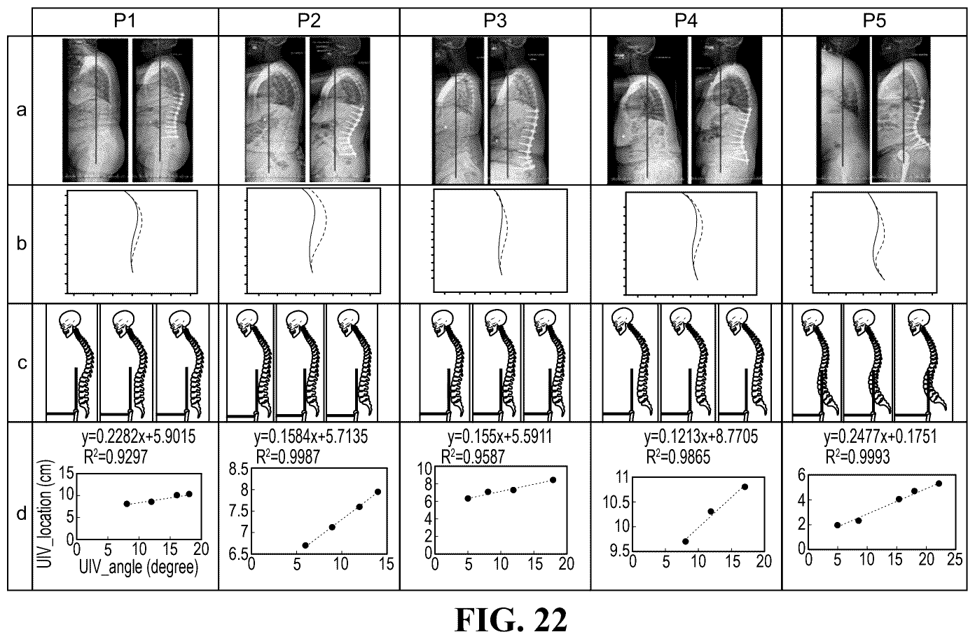

Five patients with anteriorly oriented UIV were selected with high and low Pls and different TK_Cobb angles (T1-UIV+1 Cobb angles). Their preoperative and immediate postoperative EOS images are depicted in FIG. 22(a). For each patient, a Bezier curve was fitted to actual postoperative alignment, and the corresponding musculoskeletal model was constructed. Following biomechanical analysis, the vertebral loading at UIV and UIV+1 was obtained. Then, a numerical study was performed to identify the effect of UIV location and orientation on the shear loading at UIV/UIV+1. In this section, SS and L4-S1 angles were considered as fixed values, the same as actual postoperative parameters, while other sagittal parameters were changed. Based on these changes, the location and orientation of the end point i.e., UIV were updated and vertebral loading of the resultant alignment was collected. It was observed that for a certain UIV orientation, changing UIV location resulted in UIV/UIV+1 shear force in different directions. From the analytical perspective, it means that in some alignments Fₘsinθₘ is greater than sinα, and the vice versa. Therefore, interpolation between UIV locations provided us with an optimal UIV location for the certain UIV angle. The optimal UIV locations corresponding to the actual postoperative UIV orientations for the five selected patients are represented in FIG. 22(b). There are some significant differences between the actual and optimal UIV locations, which may explain the reasons for PJK development. The blue dots show inflection points (IP) in optimal alignments. Although the proposed optimal alignments have the same UIV orientations and SS values compared to the actual postoperative ones, other sagittal parameters like L4-S1 angle, lordosis apex, and IP are different. Another optimization procedure was applied to modify the alignments based on different realignment criteria, as discussed below.

By repeating the procedure for other diverse UIV orientations, different sets of optimal UIV location and orientation were identified as shown in FIG. 22(c). In this figure, UIV orientation, and location are in degree and cm, respectively. To obtain a relationship between UIV location and orientation for each patient, the values resulting zero-shear loading (0±10N) at UIV/UIV+1 junction were correlated as illustrated in FIG. 21(d). The high R-squared values show the linearity of this correlation.

### Comparison of Different Alignment Criteria

### 3.3.1 Original Musculoskeletal Models:

The musculoskeletal analyses for models constructed from alignments based on different pre-planning strategies were performed for three randomly selected patients from PJK group. Compensatory mechanism at TK and pelvis were considered for all models. The optimal UIV location and orientation were not considered in the construction of original musculoskeletal models. Some spinopelvic parameters like L4_S1 and SS, posteriorly orientated UIV angle with respect to vertical line, FH-UIV distance, UIV compressive loading, and UIV/UIV+1 shear loading on UIV+1 local coordinate system are reported in Table 2. For GAP-recommended alignments, most of the shear values at that level are high and positive.

TABLE 2
Spinopelvic parameters and calculated shear loading at UIV/UIV +
1 for original models based on Roussouly (R-based),
age-adjusted (AA-based), and GAP (Gap-based) strategies

| | | | | | | | UIV/UIV + 1 | UIV |
|---|---|---|---|---|---|---|---|---|
| | | | SS | L4_S1 | UIV_angle | FH_UIV | shear | Compression |
| Patients | PI | | (degree) | (degree) | (degree) | (cm) | (N) | (N) |
| 1 | 42 | R-based | 22.2 | 32.7 | 9 | 11.8 | 100.66 | 286.77 |
| | | AA-based | 12.9 | 18.9 | 11.5 | 12.2 | 48.60 | 303.59 |
| | | Gap-based | 33.7 | 33.6 | 65 | 10.9 | 139.97 | 183.88 |
| 2 | 41 | R-based | 22.8 | 27.6 | 1.6 | 14.4 | 67.12 | 294.59 |
| | | AA-based | 15 | 21.6 | 8.8 | 12.6 | 75.47 | 280.35 |
| | | Gap-based | 32 | 32 | 36.4 | 10.6 | 91.84 | 307.99 |
| 3 | 55 | R-based | 33 | 41 | 1.8 | 10.9 | 132.45 | 240.21 |
| | | AA-based | 38.7 | 43.6 | 18.2 | 10 | 75.03 | 219.64 |
| | | Gap-based | 41.3 | 38.5 | 47.4 | 11.6 | 45.46 | 263.52 |

### Optimal Musculoskeletal Models:

The optimal musculoskeletal models were constructed based on targeted SS and L4-S1 of different criteria along with the optimal UIV location and orientation. Comparing the shear forces at proximal junction revealed no significant difference between different optimal alignments; however, the shear loading of original alignments were very different. To understand the difference between realignment criteria, targeted alignments of three common pre-planning criteria, i.e., Roussouly, age-adjusted, and GAP for a typical patient with low PI undergoing T10-pelvis fusion surgery are shown in FIG. 5.

### Discussion

Despite several established realignment criteria, available techniques have not been succeeded in PJK mitigation; consequently, ideal alignment to minimize complications and maximize preferable outcomes has yet to be defined. Comparing the vertebral loading pre and postoperatively may help mitigate PJK. Although, by obtaining vertebral alignment from EOS images and using musculoskeletal analysis, vertebral loading preoperatively can be calculated, predicting postoperative vertebral loading is a challenging issue, which needs more attention. To predict optimal postoperative alignment, vertebral loading of preplanning alignments was compared in this study.

In order to comprehensively investigate the vertebral loading progressing PJK, we believe that even predicting immediate postoperative sagittal alignment without compensatory mechanism may be incomplete. Comparing compressive loading pre to postoperatively shows that fusion reduces the vertebral compressive loading, while is not the case for shear loading. Therefore, it is hypothesized that PJK happens for alignments with postoperative high shear loading at UIV or UIV+1. The shear loading at top and bottom surfaces of UIV+1, especially when they are on opposite directions, can cause high shear couple which tends to rotate the whole vertebra. This shear couple will be against the moment at UIV/UIV+1 intervertebral disc to maintain the static situation. However, the resisting moment will transfer into UIV and its resultant compression will add up to the uniformly distributed compression, and if the increased compressive stress is greater than UIV strength, compression fracture at UIV will be resulted. As in most cases, the developed shear couples are counter clockwise, the transferred moment along with compressive loading cause excessive compression at the anterior side of the UIV, which forms a common compression fracture site, as shown in FIG. 20(e).

Although the free body diagram in FIG. 21 illustrates the analytical relation between shear force and resultant muscle forces around UIV+1 and the correlation between UIV+1 location and orientation, the approach in this study is to numerically calculate shear forces by employing OpenSim analysis. Numerical results confirm the analytical correlation between UIV location and orientation.

SVA characterized by global alignment is used to assess truncal inclination, which relates significantly with outcome. However, regional loss of lordosis due to ASD is followed by compensatory mechanisms implying more effective parameters for a consideration like pelvis position, as a body's reaction to a deformity, and unfused segment compensation. Recently a machine-learning (ML) approach was also used to predict the postoperative unfused thoracic kyphosis and pelvic compensation in patients who underwent fusion from the lower thoracic to the sacrum. Since many of these parameters are dependent and dynamically involved constructing the overall spinal alignment, we developed a mathematical foundation based on which some spinopelvic parameters are set and the other dependent radiological parameters are calculated.

Overcorrection of lumbar lordosis (LL) was addressed as a profound risk factor for PJK, as high lumbar lordosis orientates UIV more posteriorly and locates it farther from line of gravity (LG). Therefore, to maintain sagittal balance TK compensatory mechanism triggers, imposes extra moment at UIV+1 and UIV, and causes PJK development accordingly. There are some proxies for overcorrection including overcorrection of LL>30 degrees from the preoperative measurement, and the angle of UIV-FH line to vertical axis >11.5 degrees. On the other hand, undercorrection was known to be responsible for pelvic non-responsiveness. The targeted UIV distance to LG cannot be reached due to pelvic non-response, which also can contribute to TK compensation and PJK development. To maximize the potential for pelvic response, however, overcorrection has to be applied to the carefully selected low risk patients. Therefore, identifying the middle ground between overcorrection and undercorrection to prevent PJK remains a challenge to spine surgeons. In this avenue, understanding the patient's deformity at baseline and applying a technique to identify an optimal patient-specific correction can be very beneficial.

Along with the idea of avoiding UIV to locate and orient more posteriorly to femoral head axis, this study brings about a new insight to determine the proper location and orientation for UIV, as it introduces them as correlated parameters with the optimal values in different sets. Meaning that it cannot be accurate to decide the optimal UIV location without considering its orientation and the vice versa. Based on our analytical explanation and numerical observation, we believe that overcorrection can be a proper choice when it remains alignment with anteriorly oriented T1_UIV+1 line and posteriorly oriented UIV+1 with respect to UIV+1 vertical line. To be specific, considering TK compensatory mechanism and pelvic responsiveness, an optimal set of UIV location and orientation is proposed to achieve minimal shear loading at proximal junction. Furthermore, musculoskeletal models based on different realignment criteria (global alignment and proportion (GAP), Roussouly-type, and age-adjusted alignment) with the proposed optimal UIV positioning (location and orientation) were created and their junctional vertebral loading were compared. Among different realignment criteria, there is a controversial opinion regarding GAP. It is believed that GAP provides lordosis overcorrection, and observation shows that overcorrection is a risk factor for PJK development. Since the main reason for such observational results are yet to be clear, investigations on GAP failing to achieve ideal realignment are not confirmed. It is well known that GAP provides high lordosis, which mostly relates to lower lordosis part (L4-S1). This moves the spine to the anterior side and causes the need for sharp changes to maintain SVA in the proper range. Overall, GAP increases UIV orientation, while decreases UIV location. However, as it was shown in this study, in order to have a low-shear alignment, the UIV location and orientation should be proportional, such that for higher UIV orientation that GAP provides, the UIV location should be high as well, or the vise versa. The comparative study between different alignments elucidates how sacrum, inflection point, and UIV are positioned differently between three strategies for this case. The compensated posture is considered for all alignments. As it is shown in FIG. 5, pelvis tilt (PT) is increasing from age-adjusted to Roussouly and GAP. This is consistent with the position of inflexion point (IP) (blue circle), which moves caudally based on the same order. UIV position, however, has minimal distance to FH with the greatest orientation in GAP, and medium and larger in Roussouly and age-adjusted, respectively.

Utilizing mathematical and musculoskeletal analyses, postoperative alignment based on different was predicted pre-planning strategies. To obtain the minimal shear loading at proximal junction, the UIV+1 orientation and location is strongly related, which helps surgeons to locate UIV in a proper distance from femoral head vertical line based on the UIV angle. Therefore, using an optimal set of UIV location and orientation, as well as a desired pre-planning strategy, shear loading at proximal junction can be minimized and the risk of PJK development can be mitigated accordingly.

The foregoing discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which may be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements.

Of course, the above described embodiments are intended to be illustrative only and in no way limiting. The described embodiments are susceptible to many modifications of form, arrangement of parts, details and order of operation. The disclosure is intended to encompass all such modification within its scope, as defined by the claims.

What is claimed is:

1. A computer-implemented method for generating a patient-specific alignment for spinal deformity correction surgery, the method comprising:
upon processing at least one pre-operative image of a patient:
obtaining a plurality of spinopelvic parameters;
constructing at least one computer model of the patient;
computing metrics of muscle expenditure and/or vertebral loading based on the at least one model; and
generating a proposed alignment based on the computed metrics.

2. The method of claim 1, wherein the at least one pre-operative image includes a sagittal view image.

3. The method of claim 1, wherein the proposed alignment includes at least one of perfect alignment, an optimal alignment, and a realistic alignment.

4. The method of claim 1, further comprising:
simulating a plurality of possible sagittal alignments.

5. The method of claim 4, wherein the plurality of possible sagittal alignments correspond to realignment criteria including at least one of Roussouly, SRS-Schwab, age-adjusted, global alignment and proportion (GAP), segmental alignment and T4-L1 axis.

6. The method of claim 1, further comprising:
generating a prediction of post-operative mechanical complication.

7. The method of claim 6, wherein the post-operative mechanical complication includes at least one of proximal junctional kyphosis (PJK), proximal junctional failure (PJF), distal junctional kyphosis (DJK), and adjacent segment disease.

8. The method of claim 1, wherein the constructing at least one computer model includes fitting a parametric curve onto a spine curvature profile.

9. The method of claim 1, wherein the metrics of vertebral loading are computed at UIV and UIV+1 levels.

10. The method of claim 1, wherein the metrics of vertebral loading include metrics of compression and/or shear loading.

11. The method of claim 1, further comprising:
generating a risk factor for a particular alignment based on finite element analysis applied to the at least one computer model.

12. The method of claim 1, wherein the at least one image includes an x-ray image.

13. The method of claim 12, wherein the at least one image includes bi-planar x-ray images.

14. The method of claim 1, wherein the at least one image includes a CT image.

15. The method of claim 1, wherein the at least one computer model includes a 3D musculoskeletal model.

16. The method of claim 1, wherein the at least one computer model includes a finite element model.

17. The method of claim 1, further comprising:
presenting a visualization of the proposed alignment.

18. The method of claim 1, further comprising:
receiving user input corresponding to a desired alignment or a change to the proposed alignment.

19. The method of claim 1, further comprising:
generating instructions for bending a fusion rod based on the proposed alignment.

20. The method of claim 1, further comprising:
generating instructions for determining screw type and placement based on the proposed alignment.

21. The method of claim 1, further comprising:
computing a pelvic compensation.

22. The method of claim 1, further comprising:
generating at least a portion of a surgical plan.

23. A computer-implemented system for generating a patient-specific alignment for spinal deformity correction surgery, the system comprising:
at least one processor;
memory in communication with the at least one processor;
software code stored in the memory, which when executed at the at least one processor causes the system to:
upon processing at least one pre-operative image of a patient:
obtain a plurality of spinopelvic parameters;
construct at least one computer model of the patient;
compute metrics of muscle expenditure and/or vertebral loading based on the at least one model; and
generate a proposed alignment based on the computed metrics.

24. A computer-implemented method for assessing a patient-specific alignment for spinal deformity correction surgery, the method comprising:
receiving a proposed alignment for a patient;
upon processing at least one pre-operative image of the patient:
obtaining a plurality of spinopelvic parameters;
constructing at least one computer model of the patient;
generating an assessment of the proposed alignment using the at least one model; and
outputting a signal based on the assessment.

25. The method of claim 24, wherein said generating the assessment includes computing metrics of muscle expenditure and/or vertebral loading for the proposed alignment based on the at least one model.

26. The method of claim 24, wherein said generating the assessment includes generating a risk factor for a particular alignment.

27. The method of claim 24, wherein said outputting the signal includes presenting at least a portion of the assessment to a user via a user interface.

28. The method of claim 24, further comprising:
generating a modification to the proposed alignment based on the signal.

29. A computer-implemented system for assessing a patient-specific alignment for spinal deformity correction surgery, the system comprising:
at least one processor;
memory in communication with the at least one processor;
software code stored in the memory, which when executed at the at least one processor causes the system to:
receive a proposed alignment for a patient;
upon processing at least one pre-operative image of the patient:
obtain a plurality of spinopelvic parameters;
construct at least one computer model of the patient;
generate an assessment of the proposed alignment using the at least one model; and
output a signal based on the assessment.
